SEQUENCE LISTING (30 sequences)

SEQ ID NO: 1 - protein, 206 aa, Staphylococcus aureus
MKKWTNRLMTIAGVVLILVAAYLFSKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 2 - DNA, 618 bp, Staphylococcus aureus
atgaaaaaatggacaaatcgattaatgacaatcgctggtgtggtacttatcctagtggcagcatatttgtttgctaaaccacatatcgataattatcttcacgataaagataaagatgaaaagattgaacaatatgataaaaatgtaaaagaacaggcgagtaaagataaaaagcagcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaa

SEQ ID NO: 3 - protein, 181 aa, Artificial Sequence
  Truncated sortase variant
KPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 4 - DNA, 543 bp, Artificial Sequence
  DNA sequence encoding truncated sortase variant
aaaccacatatcgataattatcttcacgataaagataaagatgaaaagattgaacaatatgataaaaatgtaaaagaacaggcgagtaaagataaaaagcagcaagctaaacctcaaattccgaaagataaatcgaaagtggcaggctatattgaaattccagatgctgatattaaagaaccagtatatccaggaccagcaacacgtgaacaattaaatagaggtgtaagctttgcagaagaaaatgaatcactagatgatcaaaatatttcaattgcaggacacactttcattggccgtccgaactatcaatttacaaatcttaaagcagccaaaaaaggtagtatggtgtactttaaagttggtaatgaaacacgtaagtataaaatgacaagtataagaaatgttaagcctacagctgtaggagttctagatgaacaaaaaggtaaagataaacaattaacattaattacttgtgatgatcttaatcgggagacaggcgtttgggaaacacgtaaaatcttggtagctacagaagtcaaa

SEQ ID NO: 5 - protein, 7 aa, Homo sapiens
LFINEGK

SEQ ID NO: 6 - protein, 10 aa, Homo sapiens
NTWNLGNNAK

SEQ ID NO: 7 - protein, 16 aa, Homo sapiens
DGQVIISGSGVTIESK

SEQ ID NO: 8 - protein, 16 aa, Homo sapiens
EGLTLPVPFNILPSPK

SEQ ID NO: 9 - protein, 7 aa, Homo sapiens
IPISQGK

SEQ ID NO: 10 - protein, 9 aa, Homo sapiens
ILNKPVGLK

SEQ ID NO: 11 - protein, 20 aa, Homo sapiens
ELEVPVHTGPNSQKTADLTR

SEQ ID NO: 12 - protein, 8 aa, Homo sapiens
GSRSQIPR

SEQ ID NO: 13 - protein, 8 aa, Homo sapiens
ILSAQGCK

SEQ ID NO: 14 - protein, 9 aa, Homo sapiens
NLSPGFNFR

SEQ ID NO: 15 - protein, 9 aa, Homo sapiens
NYMMSNGYK

SEQ ID NO: 16 - protein, 15 aa, Homo sapiens
FQGKWGTVCDDNFSK

SEQ ID NO: 17 - protein, 6 aa, Homo sapiens
LFGGKK

SEQ ID NO: 18 - protein, 16 aa, Homo sapiens
DIKPDNVLLDVNGHIR

SEQ ID NO: 19 - protein, 7 aa, Homo sapiens
GALKQNK

SEQ ID NO: 20 - protein, 15 aa, Homo sapiens
NSQGSEMFGDDDKRR

SEQ ID NO: 21 - protein, 9 aa, Homo sapiens
KENSFEMQR

SEQ ID NO: 22 - protein, 13 aa, Homo sapiens
RQAMKEMSIDQAR

SEQ ID NO: 23 - protein, 15 aa, Homo sapiens
LLLSVLPQHVAMEMK

SEQ ID NO: 24 - protein, 8 aa, Homo sapiens
TIELQMKK

SEQ ID NO: 25 - protein, 9 aa, Homo sapiens
CSVNNQQSK

SEQ ID NO: 26 - protein, 14 aa, Homo sapiens
NQELCQVAVEKSPK

SEQ ID NO: 27 - protein, 147 aa, Artificial Sequence
  Truncated sortase variant
QAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 28 - DNA, 441 bp, Artificial Sequence
  DNA sequence encoding truncated sortase variant
caagctaaacctcaaattccgaaagataaatcgaaagtggcaggctatattgaaattccagatgctgatattaaagaaccagtatatccaggaccagcaacacgtgaacaattaaatagaggtgtaagctttgcagaagaaaatgaatcactagatgatcaaaatatttcaattgcaggacacactttcattggccgtccgaactatcaatttacaaatcttaaagcagccaaaaaaggtagtatggtgtactttaaagttggtaatgaaacacgtaagtataaaatgacaagtataagaaatgttaagcctacagctgtaggagttctagatgaacaaaaaggtaaagataaacaattaacattaattacttgtgatgatcttaatcgggagacaggcgtttgggaaacacgtaaaatcttggtagctacagaagtcaaa

SEQ ID NO: 29 - protein, 148 aa, Artificial Sequence
  truncated sortase variant
MQAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 30 - DNA, 444 bp, Artificial Sequence
  DNA sequence encoding truncated sortase variant
atgcaagctaaacctcaaattccgaaagataaatcgaaagtggcaggctatattgaaattccagatgctgatattaaagaaccagtatatccaggaccagcaacacgtgaacaattaaatagaggtgtaagctttgcagaagaaaatgaatcactagatgatcaaaatatttcaattgcaggacacactttcattggccgtccgaactatcaatttacaaatcttaaagcagccaaaaaaggtagtatggtgtactttaaagttggtaatgaaacacgtaagtataaaatgacaagtataagaaatgttaagcctacagctgtaggagttctagatgaacaaaaaggtaaagataaacaattaacattaattacttgtgatgatcttaatcgggagacaggcgtttgggaaacacgtaaaatcttggtagctacagaagtcaaa

(19) United States
(12) Patent Application Publication
(10) Pub. No.: US 2023/0145118 A1
(43) Pub. Date: May 11, 2023

(54) Modified Red Blood Cells and Uses Thereof for Delivering Agents

(71) Applicant: Westlake Therapeutics (Hangzhou) Co. Limited, Hangzhou, Zhejiang (CN)

(72) Inventors: Xiaofei Gao, Hangzhou (CN); Yanjie Huang, Hangzhou (CN); Jia Dong, Hangzhou (CN); Xiaoqian Nie, Hangzhou (CN)

(21) Appl. No.: 17/906,435

(22) PCT Filed: Mar. 19, 2021
(86) PCT No.: PCT/CN2021/081838

§ 371 Date: Sep. 15, 2022

(30) Foreign Application Priority Data
Mar. 20, 2020 (CN) PCT/CN2020/080476

(51) Int. Cl. A61K 47/69; C12N 5/00; C12N 5/078; C12N 9/52; A61K 38/48; C12N 9/96; A61K 35/18; A61P 31/14

(52) U.S. Cl. CPC A61K 47/6901; C12N 5/0006; C12N 5/0641; C12N 9/52; C12Y 304/2207; A61K 38/4813; C12Y 304/17023; C12N 9/96; A61K 35/18; A61P 31/14; C07K 2319/30

(57) ABSTRACT

A red blood cell (RBC) having an agent linked thereto, wherein the agent is linked to at least one endogenous, non-engineered membrane protein of the RBC by a sortase-mediated reaction, preferably by a sortase-mediated glycine conjugation and/or a sortase-mediated lysine side chain ε-amino group conjugation, which may occurring at least on glycine (n) and/or lysine ε-amino group at internal sites of the extracellular domain of at least one endogenous, non-engineered membrane protein, preferably n being 1 or 2, as well as the use of the RBC for delivering drugs and probes.

### TECHNICAL FIELD

The present disclosure relates generally to modified red blood cells (RBCs), and more particularly to covalently modified RBCs and use of the same for delivering drugs and probes.

### BACKGROUND

Recent development in drug delivery systems for prolonging drug retention time in treating varieties of human diseases has attracted much attention. However, many of the systems still suffer from various challenges and limitations such as poor stability, unwanted toxicity and immune responses[1]. Red blood cells (RBCs), the most common cell type in the human body, have been widely investigated as an ideal in vivo drug delivery system for over three decades due to their unique biological properties: (i) widespread circulation range throughout the body; (ii) good biocompatibility as a biological material with long in vivo survival time; (iii) large surface to volume ratio; (iv) no nucleus, mitochondria and other cellular organelles.

RBCs have been developed as drug delivery carriers by direct encapsulation, noncovalent attachment of foreign peptides, or through installation of proteins by fusion to antibodies specific for RBC surface proteins. It has been demonstrated that such modified RBCs have limitations for applications in vivo. For instance, encapsulation will disrupt cell membranes which subsequently affect in vivo survival rates of engineered cells. In addition, the non-covalent attachment of polymeric particles to RBCs dissociates readily, and the payloads will be degraded shortly in vivo.

Bacterial sortases are transpeptidases capable of modifying proteins in a covalent and site-specific manner[2]. Wild type sortase A from Staphylococcus aureus (wt SrtA) recognizes an LPXTG motif and cleaves between threonine and glycine to form a covalent acyl-enzyme intermediate between the enzyme and the substrate protein. This intermediate is resolved by a nucleophilic attack by a peptide or protein normally with three consecutive glycine residues (3×glycines, G₃) at the N-terminus. Previous studies have genetically overexpressed a membrane protein KELL with LPXTG motif on its C-terminus on RBCs, which can be attached to the N terminus of 3×glycines—or G_{(n≥3)}—modified proteins/peptides by using wt SrtA[3]. These RBCs carrying drugs have shown efficacy in treating diseases on animal models. However, this requires steps of engineering hematopoietic stem or progenitor cells (HSPCs) and differentiating these cells into mature RBCs, which significantly limits the application.

Accordingly, there is still a need in the art for an improved RBC delivering system.

### SUMMARY

In one general aspect, provided is a red blood cell (RBC) having an agent linked thereto, wherein the agent is linked to at least one endogenous, non-engineered membrane protein of the RBC by a sortase-mediated reaction, preferably by a sortase-mediated glycine conjugation and/or a sortase-mediated lysine side chain ε-amino group conjugation. In some embodiments, the sortase-mediated glycine conjugation and/or the sortase-mediated lysine side chain ε-amino group conjugation occur at least on glycine₍ₙ₎ and/or lysine ε-amino group at internal sites of the extracellular domain of the at least one endogenous, non-engineered membrane protein, preferably n being 1 or 2.

In some embodiments, the RBC has not been genetically engineered to express a protein comprising a sortase recognition motif or a nucleophilic acceptor sequence, and preferably the RBC is a natural RBC such as a natural human RBC.

In some embodiments, the sortase is capable of mediating a glycine₍ₙ₎ conjugation and/or a lysine side chain ε-amino group conjugation, preferably at internal sites of the extracellular domain of the at least one endogenous, non-engineered membrane protein, preferably n being 1 or 2.

In some embodiments, the sortase is a Sortase A (SrtA) such as a Staphylococcus aureus transpeptidase A variant (mgSrtA). For example, the mgSrtA comprises or consists essentially of or consists of an amino acid sequence having at least 60% identity to an amino acid sequence as set forth in SEQ ID NO: 3.

In some embodiments, the agent, before being linked to the RBC, comprises a sortase recognition motif on its C-terminus.

In some embodiments, the sortase recognition motif comprises or consists essentially of or consists of an amino acid sequence selecting from a group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid.

In some embodiments, the agent comprises a binding agent, a therapeutic agent, or a detection agent, including for example a protein, a peptide such as an extracellular domain of oligomeric Angiotensin-converting enzyme 2 (ACE2), an antibody or its functional antibody fragment, an antigen or epitope such a tumor antigen, a MHC-peptide complex, a drug such as a small molecule drug (e.g., an antitumor agent such as a chemotherapeutic agent), an enzyme (e.g., a functional metabolic or therapeutic enzyme), a hormone, a cytokine, a growth factor, an antimicrobial agent, a probe, a ligand, a receptor, an immunotolerance-inducing peptide, a targeting moiety, a prodrug or any combination thereof.

In some embodiments, the agent linked to the at least one endogenous, non-engineered membrane protein on the surface of the BRC comprises a structure of A¹-LPXT-P¹, in which LPXT is linked to a glycine₍ₙ₎ in P¹, and/or a structure of A¹-LPXT-P², in which LPXT is linked to the side chain ε-amino group of lysine in P², wherein n is preferably 1 or 2, A¹ represents the agent, P¹ and P² independently represent the extracellular domain of the at least one endogenous, non-engineered membrane protein, and X represents any amino acids.

In another aspect, provided is a red blood cell (RBC) having an agent linked to at least one endogenous, non-engineered membrane protein on the surface of the BRC, wherein the agent linked to the at least one endogenous, non-engineered membrane protein comprises a structure of A¹-LPXT-P¹, in which LPXT is linked to a glycine₍ₙ₎ in P¹, and/or a structure of A¹-LPXT-P², in which LPXT is linked to the side chain ε-amino group of lysine in P², wherein n is preferably 1 or 2, A¹ represents the agent, P¹ and P² independently represent the at least one endogenous, non-engineered membrane protein, and X represents any amino acids. In some embodiments, the linking occurs at least on glycine₍ₙ₎ and/or lysine ε-amino group at internal sites of the extracellular domain of the at least one endogenous, non-engineered membrane protein, preferably n being 1 or 2.

In another general aspect, provided is a method for covalently modifying at least one endogenous, non-engineered membrane protein of a red blood cell (RBC), comprising contacting the RBC with a sortase substrate that comprises a sortase recognition motif and an agent, in the presence of a sortase under conditions suitable for the sortase to conjugate the sortase substrate to the at least one endogenous, non-engineered membrane protein of the RBC by a sortase-mediated reaction, preferably by a sortase-mediated glycine conjugation and/or a sortase-mediated lysine side chain ε-amino group conjugation. In some embodiments, the sortase-mediated glycine conjugation and/or the sortase-mediated lysine side chain ε-amino group conjugation occur at least on glycine₍ₙ₎ and/or lysine ε-amino group at internal sites of the extracellular domain of the at least one endogenous, non-engineered membrane protein, preferably n being 1 or 2.

In some embodiments, the RBC has not been genetically engineered to express a protein comprising a sortase recognition motif or a nucleophilic acceptor sequence, and preferably the RBC is a natural RBC such as a natural human RBC.

In some embodiments, the sortase is capable of mediating a glycine₍ₙ₎ conjugation and/or a lysine side chain ε-amino group conjugation, preferably at internal sites of the extracellular domain of the at least one endogenous, non-engineered membrane protein, preferably n being 1 or 2.

In some embodiments, the sortase is a Sortase A (SrtA) such as a Staphylococcus aureus transpeptidase A variant (mgSrtA). For example, the mgSrtA comprises or consists essentially of or consists of an amino acid sequence having at least 60% identity to an amino acid sequence as set forth in SEQ ID NO: 3.

In some embodiments, the sortase substrate comprises the sortase recognition motif on its C-terminus.

In some embodiments, the sortase recognition motif comprises or consists essentially of or consists of an amino acid sequence selecting from a group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid.

In some embodiments, the agent comprises a binding agent, a therapeutic agent, or a detection agent, including for example a protein, a peptide such as an extracellular domain of oligomeric ACE2, an antibody or its functional antibody fragment, an antigen or epitope such a tumor antigen, a MHC-peptide complex, a drug such as a small molecule drug (e.g., an antitumor agent such as a chemotherapeutic agent), an enzyme (e.g., a functional metabolic or therapeutic enzyme), a hormone, a cytokine, a growth factor, an antimicrobial agent, a probe, a ligand, a receptor, an immunotolerance-inducing peptide, a targeting moiety, a prodrug or any combination thereof.

In some embodiments, the covalently modified at least one endogenous, non-engineered membrane protein on the surface of the BRC comprises a structure of A¹-LPXT-P¹, in which LPXT is linked to a glycine₍ₙ₎ in P¹, and/or a structure of A¹-LPXT-P², in which LPXT is linked to the side chain ε-amino group of lysine in P², wherein n is preferably 1 or 2, A¹ represents the agent, P¹ and P² independently represent the at least one endogenous, non-engineered membrane protein, and X represents any amino acids.

In another aspect, provided is a red blood cell (RBC) obtained by any of claims 13-22.

In another aspect, provided is a composition comprising the red blood cell having an agent linked thereto of the present disclosure and optionally a physiologically acceptable carrier.

In another aspect, provided is a composition comprising a sortase, a sortase substrate that comprises a sortase recognition motif and an agent, and optionally a physiologically acceptable carrier, wherein the sortase is capable of mediating a glycine₍ₙ₎ conjugation and/or a lysine side chain ε-amino group conjugation, preferably at internal sites of the extracellular domain of the at least one endogenous, non-engineered membrane protein, preferably n being 1 or 2.

In some embodiments, the sortase is a Sortase A (SrtA) such as a Staphylococcus aureus transpeptidase A variant (mgSrtA). For example, the mgSrtA comprises or consists essentially of or consists of an amino acid sequence having at least 60% identity to an amino acid sequence as set forth in SEQ ID NO: 3.

In some embodiments, the sortase substrate comprises the sortase recognition motif on its C-terminus.

In some embodiments, the sortase recognition motif comprises or consists essentially of or consists of an amino acid sequence selecting from a group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid.

In some embodiments, the agent comprises a binding agent, a therapeutic agent, or a detection agent, including for example a protein, a peptide such as an extracellular domain of oligomeric ACE2, an antibody or its functional antibody fragment, an antigen or epitope such a tumor antigen, a MHC-peptide complex, a drug such as a small molecule drug (e.g., an antitumor agent such as a chemotherapeutic agent), an enzyme (e.g., a functional metabolic or therapeutic enzyme), a hormone, a cytokine, a growth factor, an antimicrobial agent, a probe, a ligand, a receptor, an immunotolerance-inducing peptide, a targeting moiety, a prodrug or any combination thereof.

In some embodiments, upon contacting red blood cells in vivo, the sortase conjugates the sortase substrate to at least one endogenous, non-engineered membrane protein of the red blood cells by a sortase-mediated reaction, preferably by a sortase-mediated glycine conjugation and/or a sortase-mediated lysine side chain conjugation.

In some embodiments, the sortase-mediated glycine conjugation and/or the sortase-mediated lysine side chain ε-amino group conjugation occur at least on glycine₍ₙ₎ and/or lysine ε-amino group at internal sites of the extracellular domain of the at least one endogenous, non-engineered membrane protein, preferably n being 1 or 2.

In some embodiments, the at least one endogenous, non-engineered membrane protein conjugated with the sortase substrate comprises a structure of A¹-LPXT-P¹, in which LPXT is linked to a glycine₍ₙ₎ in P¹, and/or a structure of A¹-LPXT-P², in which LPXT is linked to the side chain ε-amino group of lysine in P², wherein n is preferably 1 or 2, A¹ represents the agent, P¹ and P² independently represent the at least one endogenous, non-engineered membrane protein, and X represents any amino acids.

In some embodiments, the sortase has been further modified to enhance its stabilization in circulation and/or reduce its immunogenicity. For example, the sortase has been PEGylated and/or linked to an Fc fragment.

In another aspect, provided is a method for diagnosing, treating or preventing a disorder, condition or disease in a subject in need thereof, comprising administering the red blood cell or the composition as described in the present disclosure to the subject.

In some embodiments, the disorder, condition or disease is selected from a group consisting of tumors or cancers, metabolic diseases, bacterial infections, virus infections such as a coronavirus infection for example SARS-COV or SARS-COV-2 infection, autoimmune diseases and inflammatory diseases.

In another aspect, provided is a method of delivering an agent to a subject in need thereof, comprising administering the red blood cell or the composition as described in the present disclosure to the subject.

In another aspect, provided is a method of increasing the circulation time or plasma half-life of an agent in a subject, comprising providing a sortase substrate that comprises a sortase recognition motif and an agent, and conjugating the sortase substrate in the presence of a sortase under conditions suitable for the sortase to conjugate the sortase substrate to the at least one endogenous, non-engineered membrane protein of a red blood cell by a sortase-mediated reaction, preferably by a sortase-mediated glycine conjugation and/or a sortase-mediated lysine side chain ε-amino group conjugation. In some embodiments, the method further comprises administering the conjugated red blood cells to a subject, e.g., directly into the circulatory system, e.g., intravenously.

In some embodiments, the sortase-mediated glycine conjugation and/or the sortase-mediated lysine side chain ε-amino group conjugation occur at least on glycine₍ₙ₎ and/or lysine ε-amino group at internal sites of the extracellular domain of the at least one endogenous, non-engineered membrane protein, preferably n being 1 or 2.

In another aspect, provided is use of the red blood cell or the composition as described herein in the manufacture of a medicament for diagnosing, treating or preventing a disorder, condition or disease, or a diagnostic agent for diagnosing a disorder, condition or disease or for delivering an agent. In some embodiments, the disorder, condition or disease is selected from a group consisting of tumors or cancers, metabolic diseases, bacterial infections, virus infections such as coronavirus infection for example SARS-COV or SARS-COV-2 infection, autoimmune diseases and inflammatory diseases. In some embodiments, the medicament is a vaccine.

In another aspect, provided is a red blood cell or composition of the present disclosure for use in diagnosing, treating or preventing a disorder, condition or disease in a subject in need thereof. In some embodiments, the disorder, condition or disease is selected from a group consisting of tumors or cancers, metabolic diseases, bacterial infections, virus infections such as coronavirus infection for example SARS-COV or SARS-COV-2 infection, autoimmune diseases and inflammatory diseases.

### BRIEF DESCRIPTION OF THE DRAWINGS

In the drawings, embodiments of the present disclosure are illustrated by way of example. It is to be expressly understood that the description and drawings are only for the purpose of illustration and as an aid to understanding, and are not intended as a definition of the limits of the invention.

FIGS. 1A-1K show the labeling efficiency of peptides and proteins on the surface of natural mouse or human RBCs by wild type sortase (wtSrtA) and mutant sortase (mg SrtA).

FIGS. 1A and 1B. 10⁹/mL mouse (FIG. 1A) or human (FIG. 1B) RBCs were incubated with 500 μM biotin-LPETG with or without 40 μM wild type (wt) SrtA or mg SrtA for 2 hrs at 4° C. After the enzymatic reaction, the labeling efficacy was detected by incubating RBCs with PE-conjugated streptavidin and analyzed by flow cytometry. Histograms show biotin signals on the surface of RBCs labeled with or without mg or wt sortase. Red: mg sortase; blue: wt sortase; orange: no sortase.

FIG. 1C. 10⁹/mL of mouse RBCs were incubated with 8 μM biotin-LPETG peptides and 40 μM mg or wt SrtA for 2 hrs at 37° C. The labeling efficacy was analyzed by immunoblotting with Streptavidin-HRP. Hemoglobin Subunit Alpha 1, HBA1, was used as the loading control.

FIG. 1D. 10⁹/mL of mouse RBCs were processed for the enrichment of membrane proteins by ultracentrifugation. Significant enrichment of membrane proteins was detected by Western-blotting of an RBC membrane protein Band 3 encoded by Slc4a1 gene.

FIG. 1E. 10⁹/mL of mouse RBCs were biotin-labeled by mg SrtA and subjected to the membrane protein enrichment. Western-blot results showed a significant increase in biotin signals after the enrichment step compared to that of unenriched samples.

FIG. 1F. 10⁹ mouse RBCs were sortagged with biotin-LPETG by mg SrtA or wt SrtA. After sortagging, labeled RBCs were stained with DiR dye and injected intravenously into the mice. Mice were bled at 24 h post transfusion. Blood samples were incubated with FITC-conjugated Streptavidin at 37° C. for 1 hour for the detection of biotin signals and washed three times before being analyzed by flow cytometry. DiR positive cells were selected for analyzing the percentage of RBCs with biotin signals.

FIG. 1G. Mice were bled at indicated days post transfusion. DiR positive cells indicate the percentage of transfused RBCs in the circulation.

FIG. 1H. DiR positive RBCs from the blood samples of the above experiments were analyzed for the percentage of biotin positive cells.

FIG. 1I. At day 4 post injection, blood samples were analyzed by imaging flow cytometry for the sortagging of biotin on RBCs. Blood samples were incubated with FITC-conjugated Streptavidin at 37° C. for 1 hour for the detection of biotin signals and washed three times before being analyzed by flow cytometry.

FIG. 1J. 10⁹/mL mouse RBCs were sortagged with 100 μM eGFP-LPETG by mg SrtA or wt SrtA at 37° C. for 2 h. The efficacy of conjugation was analyzed by flow cytometry. Histograms show biotin signals on the surface of RBCs labeled with or without mg or wt sortase. Red: no sortase; blue: mg sortase; orange: wt sortase.

FIG. 1K. 10⁹ eGFP-sortagged mouse RBCs were stained by DiR dye and injected intravenously into the mice. At day 7 post injection, the mice were bled and the blood samples were analyzed by imaging flow cytometry for eGFP signals on the surface of RBCs.

FIG. 2 shows intravenous injection of OT-1-RBCs induces immunotolerance in OT-1 TCR T cells in vivo.

FIG. 2A. 10⁶ CD8⁺ T cells purified from CD45.1 OT-1 TCR transgenic mice were intravenously injected into CD45.2 recipient mice. After 24 hrs, 2×10⁹ mouse RBCs were labeled with or without OT-1 peptides mediated by mg SrtA and transfused into the recipient mice, which will be challenged with OT-1 peptide with complete freund's adjuvant (CFA). At day 15, these mice were euthanized and subjected to spleen harvest.

FIG. 2B. Suspended cells isolated from spleen were analyzed by flow cytometry. CD8⁺ T cells were first selected out for analyzing the percentage of CD45.1+ T cells, which demonstrates the survival of adoptively transferred OT-1 TCR CD8+ T cells. CD45.1+ CD8+ T cells were further analyzed for the expression of PD1 and CD44. CD45.2: membrane protein expressed on the surface of many hematopoietic cells used for indicating endogenous T cells in this experiment. CD44: marker for T cell activation; PD-1: marker for cell apoptosis and exhaustion.

FIG. 3 shows that SARS-CoV-2 enters host cells through binding with ACE2 by its S protein.

FIG. 4 shows red blood cell (RBC) with trimeric ACE2 engineered on surface.

FIG. 5 shows the labeling efficiency of ACE2-Fc-LPETG on the surface of natural RBCs.

FIG. 6 shows the life-span of the ACE2-Fc labeled RBCs in vivo.

FIG. 7 shows the inhibition of SARS-COV-2 virus by ACE2-RBC.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, unless otherwise specified, the scientific and technical terms used herein have the meanings as generally understood by a person skilled in the art. Although any methods and materials similar or equivalent to those described herein find use in the practice of the present invention, the preferred methods and materials are described herein. Accordingly, the terms defined herein are more fully described by reference to the Specification as a whole.

As used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skills in the art.

As used herein, the term “consisting essentially of” in the context of an amino acid sequence is meant the recited amino acid sequence together with additional one, two, three, four or five amino acids at the N- or C-terminus.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

As used herein, the terms “patient”, “individual” and “subject” are used in the context of any mammalian recipient of a treatment or composition disclosed herein. Accordingly, the methods and composition disclosed herein may have medical and/or veterinary applications. In a preferred form, the mammal is a human.

As used herein, the term “sequence identity” is meant to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

Recent studies have discovered mutant sortases with different specificities in motif recognition[4]. For instance, Ge et al. showed that an evolved SrtA variant (mg SrtA) is capable of recognizing the N-terminus of Gi-modified peptide, which cannot be achieved by wt SrtA[5]. In addition, membrane proteins with a single glycine at the N-terminus are much more abundant than those with 3×glycines. Ge et al. made an N-terminal sequence analysis of human membrane proteome with a predicted N-terminal glycine(s). The list of 182 proteins that contain N-terminal glycine residues after enzymatic removal of the signal peptide or the initiator methionine residue according to the previous study[7]. Among them, 176 proteins (96.70%) contain a single glycine residue at the N-terminus, 4 proteins (2.20%) contain a GG residue at the N-terminus, while only 2 proteins (1.10%) contain a G_{(n≥3)} residue at the N-terminus. None of the 182 proteins is known to be expressed on the surface of mature human red blood cells.

Herein, the present disclosure is at least partially based on a surprising finding that in spite of the absence of known N-terminal glycine(s), it is possible to conjugate a sortase substrate to at least one endogenous, non-engineered membrane protein of natural human RBC by a sortase-mediated glycine conjugation and/or a sortase-mediated lysine side chain conjugation occurring at least on glycine_{(n=1 or 2)} and lysine ε-amino group at internal sites of the extracellular domain of the at least one endogenous, non-engineered membrane protein. Without being limited by theory, it is contemplated that a non-canonical function of sortase enables conjugation of a sortase substrate to internal glycines_{(n=1 or 2)} and/or lysine side chain ε-amino group in the extracellular domain of endogenous, non-engineered membrane protein. Also, without being limited by any theory, extensive tissue-specific mRNA splicing and protein translation during erythropoiesis might lead to exposure of glycine_{(n=1 or 2)}.

The inventors therefore develop a new strategy to covalently modify endogenous, non-engineered membrane proteins of natural RBCs with peptides and/or small molecules through a sortase-mediated reaction. The technology allows for producing RBC products by directly modifying natural RBCs instead of HSPCs which are limited by their resources. Also, the modified RBCs preserve their original biological properties well and remain stable as their native state.

### Red Blood Cells (RBCs)

In some aspects, the present disclosure provides a red blood cell (RBC) having an agent linked thereto, wherein the agent is linked to at least one endogenous, non-engineered membrane protein of the RBC by a sortase-mediated reaction. In some embodiments, the agent is linked to at least one endogenous, non-engineered membrane protein through a sortase-mediated glycine conjugation and/or a sortase-mediated lysine side chain ε-amino conjugation. In some embodiments, the sortase-mediated glycine conjugation and/or the sortase-mediated lysine side chain ε-amino group conjugation occur at least on glycine₍ₙ₎ and/or lysine ε-amino group in the extracellular domain (for example at internal sites of the extracellular domain) of the at least one endogenous, non-engineered membrane protein, preferably n being 1 or 2. In some embodiments, without being limited to any theory, the sortase-mediated glycine conjugation may occur at exposed glycine_{(n=1 or 2)} of previously unreported membrane proteins due to tissue-specific mRNA splicing and protein translation during erythropoiesis. In some embodiments, the exposed glycine_{(n=1 or 2)} may be N-terminal exposed glycine_{(n=1 or 2)}. In some embodiments, the sortase-mediated lysine side chain ε-amino group conjugation occurs at ε-amino group of terminal lysine or internal lysine of the extracellular domain. In some embodiments, the sortase-mediated glycine conjugation and/or the sortase-mediated lysine side chain ε-amino group conjugation may occur at glycine₍ₙ₎ and/or lysine ε-amino group at terminal (e.g., N-terminal) and/or internal sites of the extracellular domain of at least one endogenous, non-engineered membrane protein, preferably n being 1 or 2.

Unless otherwise indicated or clearly evident from the context, where the present disclosure refers to a red blood cell (RBC), it is generally intended to mean a mature red blood cell. In certain embodiments, the RBC is a human RBC, such as a human natural RBC.

In some embodiments, the RBC is a red blood cell that has not been genetically engineered to express a protein comprising a sortase recognition motif or a nucleophilic acceptor sequence. In some embodiments the RBC has not been genetically engineered. Unless otherwise indicated or clearly evident from the context, where the present disclosure refers to sortagging red blood cells it is generally intended to mean red blood cells that have not been genetically engineered for sortagging. In certain embodiments the red blood cells are not genetically engineered.

A red blood cell is considered “not genetically engineered for sortagging” if the cell has not been genetically engineered to express a protein comprising a sortase recognition motif or a nucleophilic acceptor sequence in a sortase-catalyzed reaction.

In some embodiments, the present disclosure provides red blood cells having an agent conjugated thereto via a sortase-mediated reaction. In some embodiments, a composition comprising a plurality of such cells is provided. In some embodiments, at least a selected percentage of the cells in the composition are modified, i.e., having an agent conjugated thereto by sortase. For example, in some embodiments at least 5%, 10%, 15%, 20%, 25%, 30%, 40%, 50%, 60%, 70%, 80%, 90%, 95%, 96%, 97%, 98%, 99%, or more of the cells have an agent conjugated thereto. In some embodiments, the conjugated agent may be one or more of the agents described herein. In some embodiments, the agent may be conjugated to glycine₍ₙ₎ and/or lysine ε-amino group in one or more or all of the sequences as listed in Table 5 (e.g., SEQ ID NOs: 5-26). In some embodiments, the agent may be conjugated to glycine₍ₙ₎ and/or lysine ε-amino group in a sequence comprising SEQ ID NO: 5.

In some embodiments, the present disclosure provides a red blood cell that comprises an agent conjugated via a sortase-mediated reaction to a non-genetically engineered endogenous polypeptide expressed by the cell. In some embodiments, two, three, four, five or more different endogenous non-engineered polypeptides expressed by the cell have an agent conjugated thereto via a sortase-mediated reaction. The agents attached to different polypeptides may be the same or the cell may be sortagged with a plurality of different agents.

In some embodiments, the present disclosure provides a red blood cell (RBC) having an agent linked via a sortase mediated reaction to a glycine₍ₙ₎ or a side chain of lysine located anywhere (preferably internal sites) in an extracellular domain of at least one endogenous, non-engineered membrane protein on the surface of the BRC, wherein n is preferably 1 or 2. In some embodiments, the agent is linked to one or more (e.g., two, three, four or five) glycine₍ₙ₎ or lysine side chain ε-amino groups in or within the extracellular domain. In certain embodiment, the at least one endogenous, non-engineered membrane protein may be selected from a group consisting of the membrane proteins listed in Table 5 below or any combination thereof. In certain embodiment, the at least one endogenous non-engineered membrane protein may be selected from a group consisting of the 22 membrane proteins listed in Table 5 or any combination thereof. In some embodiments, the sortase-mediated glycine conjugation and/or the sortase-mediated lysine side chain ε-amino group conjugation may occur at glycine₍ₙ₎ and/or lysine ε-amino group in one or more or all of the sequences as listed in Table 5 (e.g., SEQ ID NOs: 5-26). In certain embodiments, the at least one endogenous non-engineered membrane protein may comprise extracellular calcium-sensing receptor (CaSR) (a parathyroid cell calcium-sensing receptor, PCaR1). In certain embodiments, the linking may be one or more or all of the modifications as shown in Table 5 below. In certain embodiments, the linking may occur on one or more positions selected from the modification positions as listed in Table 5 and any combination thereof, e.g., positions comprising G526 and/or K527 positions of CaSR; G158 and/or K162 of CD antigen CD3g; and/or G950 and/or K964 of TrpC2.

In some embodiments, without being limited to any theory, the agent may be linked to a protein selected from a group consisting of proteins listed in Tables 2, 3 and/or 4 below or any combination thereof.

In some embodiments, the present disclosure provides a red blood cell (RBC) having an agent linked to at least one endogenous, non-engineered membrane protein on the surface of the BRC. In some embodiments, the agent is linked via a sortase recognition motif to the at least one endogenous, non-engineered membrane protein. In some embodiments, the sortase recognition motif may be selected from a group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid. It can be understood that after the agent linked to the membrane protein, the last (e.g., 5^{th} from the direction of N-terminal to C-terminal) residue of the sortase recognition motif is replaced by the amino acid on which the linkage occurs, as described elsewhere herein. For example, the agent linked to the at least one endogenous, non-engineered membrane protein comprises A¹-L¹-P¹, in which L¹ is linked to a glycine₍ₙ₎ in P¹, and/or a structure of A¹-L¹-P², in which L¹ is linked to the side chain ε-amino group of lysine in P², wherein n is preferably 1 or 2; L¹ is selected from the group consisting of LPXT, LPXA, LPXS, LPXL, LPXV, LGXT, LAXT, LSXT, NPXT, MPXT, IPXT, SPXT, VPXT, YPXR, LPXT and LPXT; A¹ represents the agent; P¹ and P² independently represent the at least one endogenous, non-engineered membrane protein; and X represents any amino acids. In some embodiments, the agent linked to the at least one endogenous, non-engineered membrane protein comprises A¹-LPXT-P¹, in which LPXT is linked to a glycine₍ₙ₎ in P¹, and/or a structure of A¹-LPXT-P², in which LPXT is linked to the side chain ε-amino group of lysine in P², wherein n is preferably 1 or 2, A¹ represents the agent, P¹ and P² independently represent the at least one endogenous, non-engineered membrane protein, and X represents any amino acids. In some embodiments, P¹ and P² may be the same or different. In some embodiments, the agent is linked to one or more (e.g., two, three, four, five or more) glycine₍ₙ₎ or lysine side chain ε-amino groups in or within an extracellular domain of the at least one endogenous, non-engineered membrane protein. In certain embodiment, the at least one endogenous, non-engineered membrane protein may be selected from a group consisting of the membrane proteins listed in Table 5 below or any combination thereof. In certain embodiment, the at least one endogenous non-engineered membrane protein may be selected from a group consisting of the 22 membrane proteins listed in Table 5 or any combination thereof. In some embodiments, the sortase-mediated glycine conjugation and/or the sortase-mediated lysine side chain ε-amino group conjugation may occur at glycine₍ₙ₎ and/or lysine ε-amino group in one or more or all of the sequences as listed in Table 5 (e.g., SEQ ID NOs: 5-26). In certain embodiments, at least one endogenous non-engineered membrane protein may comprise extracellular calcium-sensing receptor (CaSR) (a parathyroid cell calcium-sensing receptor, PCaR1). In certain embodiments, the linking may be one or more or all of the modifications as shown in Table 5 below. In certain embodiments, the linking may occur on one or more positions selected from the modification positions as listed in Table 5 and any combination thereof, e.g., positions comprising G526 and/or K527 positions of CaSR; G158 and/or K162 of CD antigen CD3g; and/or G950 and/or K964 of TrpC2.

In some embodiments, genetically engineered red blood cells are modified by using sortase to attach a sortase substrate to a non-genetically engineered endogenous polypeptide of the cell. The red blood cell may, for example, have been genetically engineered to express any of a wide variety of products, e.g., polypeptides or noncoding RNAs, may be genetically engineered to have a deletion of at least a portion of one or more genes, and/or may be genetically engineered to have one or more precise alterations in the sequence of one or more endogenous genes. In certain embodiments, a non-engineered endogenous polypeptide of such genetically engineered cell is sortagged with any of the various agents described herein.

In some embodiments, the present disclosure contemplates using autologous red blood cells that are isolated from an individual to whom such isolated red blood cells, after modified in vitro, are to be administered. In some embodiments, the present disclosure contemplates using immuno-compatible red blood cells that are of the same blood group as an individual to whom such cells are to be administered (e.g., at least with respect to the ABO blood type system and, in some embodiments, with respect to the D blood group system) or may be of a compatible blood group.

### Endogenous, Non-Engineered Membrane Proteins

The terms “non-engineered, “non-genetically modified” and “non-recombinant” as used herein are interchangeable and refer to not being genetically engineered, absence of genetic modification, etc. Non-engineered membrane proteins encompass endogenous proteins. In certain embodiments, a non-genetically engineered red blood cell does not contain a non-endogenous nucleic acid, e.g., DNA or RNA that originates from a vector, from a different species, or that comprises an artificial sequence, e.g., DNA or RNA that was introduced artificially. In certain embodiments, a non-engineered cell has not been intentionally contacted with a nucleic acid that is capable of causing a heritable genetic alteration under conditions suitable for uptake of the nucleic acid by the cells.

In some embodiments, the endogenous non-engineered membrane proteins may encompass any or at least one of the membrane proteins listed in Table 5 below or any combination thereof. In certain embodiments, the endogenous non-engineered membrane proteins may encompass any or at least one of the 22 membrane proteins listed in Table 5 or any combination thereof. In certain embodiments, the endogenous non-engineered membrane proteins may encompass extracellular calcium-sensing receptor (CaSR) (a parathyroid cell calcium-sensing receptor, PCaR1).

### Sortase

Enzymes identified as “sortases” have been isolated from a variety of Gram-positive bacteria. Sortases, sortase-mediated transacylation reactions, and their use in protein engineering are well known to those of ordinary skills in the art (see, e.g., PCT/US2010/000274 (WO/2010/087994), and PCT/US2011/033303 (WO/2011/133704)). Sortases have been classified into 4 classes, designated A, B, C, and D, based on sequence alignment and phylogenetic analysis of 61 sortases from Gram-positive bacterial genomes (Dramsi S, Trieu-Cuot P, Bierne H, Sorting sortases: a nomenclature proposal for the various sortases of Gram-positive bacteria. Res Microbiol. 156(3):289-97, 2005). Those skilled in the art can readily assign a sortase to the correct class based on its sequence and/or other characteristics such as those described in Drami, et al., supra. The term “sortase A” as used herein refers to a class A sortase, usually named SrtA in any particular bacterial species, e.g., SrtA from S. aureus or S. pyogenes.

The term “sortase” also known as transamidases refers to an enzyme that has transamidase activity. Sortases recognize substrates comprising a sortase recognition motif, e.g., the amino acid sequence LPXTG. A molecule recognized by a sortase (i.e., comprising a sortase recognition motif) is sometimes termed a “sortase substrate” herein. Sortases tolerate a wide variety of moieties in proximity to the cleavage site, thus allowing for the versatile conjugation of diverse entities so long as the substrate contains a suitably exposed sortase recognition motif and a suitable nucleophile is available. The terms “sortase-mediated transacylation reaction”, “sortase-catalyzed transacylation reaction”, “sortase-mediated reaction”, “sortase-catalyzed reaction”, “sortase reaction”, “sortase-mediated transpeptide reaction” and like terms, are used interchangeably herein to refer to such a reaction. The terms “sortase recognition motif”, “sortase recognition sequence” and “transamidase recognition sequence” with respect to sequences recognized by a transamidase or sortase, are used interchangeably herein. The term “nucleophilic acceptor sequence” refers to an amino acid sequence capable of serving as a nucleophile in a sortase-catalyzed reaction, e.g., a sequence comprising an N-terminal glycine (e.g., 1, 2, 3, 4, or 5 N-terminal glycines) or in some embodiments comprising internal glycines_{(n=1 or 2)} or lysine side chain ε-amino group.

The present disclosure encompasses embodiments relating to any of the sortase classes known in the art (e.g., a sortase A, B, C or D from any bacterial species or strain). In some embodiments, sortase A is used, such as SrtA from S. aureus. In some embodiments it is contemplated to use two or more sortases. In some embodiments the sortases may utilize different sortase recognition sequences and/or different nucleophilic acceptor sequences.

In some embodiments, the sortase is a sortase A (SrtA). SrtA recognizes the motif LPXTG, with common recognition motifs being, e.g., LPKTG, LPATG, LPNTG. In some embodiments LPETG is used. However, motifs falling outside this consensus may also be recognized. For example, in some embodiments the motif comprises an ‘A’, ‘S’, ‘L’ or ‘V’ rather than a ‘T’ at position 4, e.g., LPXAG, LPXSG, LPXLG or LPXVG, e.g., LPNAG or LPESG, LPELG or LPEVG. In some embodiments the motif comprises an ‘A’ rather than a ‘G’ at position 5, e.g., LPXTA, e.g., LPNTA. In some embodiments the motif comprises a ‘G’ or ‘A’ rather than ‘P’ at position 2, e.g., LGXTG or LAXTG, e.g., LGATG or LAETG. In some embodiments the motif comprises an ‘I’ or ‘M’ rather than ‘L’ at position 1, e.g., MPXTG or IPXTG, e.g., MPKTG, IPKTG, IPNTG or IPETG. Diverse recognition motifs of sortase A are described in Pishesha et al. 2018.

In some embodiments, the sortase recognition sequence is LPXTG, wherein X is a standard or non-standard amino acid. In some embodiments, X is selected from D, E, A, N, Q, K, or R. In some embodiments, the recognition sequence is selected from LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X may be any amino acids, such as those selected from D, E, A, N, Q, K, or R in certain embodiments.

In some embodiments, the present disclosure contemplates using a variant of a naturally occurring sortase. In some embodiments, the variant is capable of mediating a glycine₍ₙ₎ conjugation and/or a lysine side chain ε-amino group conjugation, preferably at internal sites of the extracellular domain of the at least one endogenous, non-engineered membrane protein of a red blood cell, preferably n being 1 or 2. Such variants may be produced through processes such as directed evolution, site-specific modification, etc. Considerable structural information regarding sortase enzymes, e.g., sortase A enzymes, is available, including NMR or crystal structures of SrtA alone or bound to a sortase recognition sequence (see, e.g., Zong Y, et al. J. Biol Chem. 2004, 279, 31383-31389). The active site and substrate binding pocket of S. aureus SrtA have been identified. One of ordinary skills in the art can generate functional variants by, for example, avoiding deletions or substitutions that would disrupt or substantially alter the active site or substrate binding pocket of a sortase. In some embodiments, directed evolution on SrtA can be performed by utilizing the FRET (Fluorescence Resonance Energy Transfer)-based selection assay described in Chen, et al. Sci. Rep. 2016, 6 (1), 31899. In some embodiments, a functional variant of S. aureus SrtA may be those described in CN10619105A and CN109797194A. In some embodiments, the S. aureus SrtA variant can be a truncated variant with e.g. 25-60 (e.g., 30, 35, 40, 45, 50, 55, 59 or 60) amino acids being removed from N-terminus (as compared to the wild type S. aureus SrtA).

In some embodiments, a functional variant of S. aureus SrtA useful in the present disclosure may be a S. aureus SrtA variant comprising one or more mutations on amino acid positions of D124, Y187, E189 and F200 of D124G, Y187L, E189R and F200L and optionally further comprising one or more mutations of P94S/R, D160N, D165A, K190E and K196T. In certain embodiments, the S. aureus SrtA variant may comprise D124G; D124G and F200L; P94S/R, D124G, D160N, D165A, K190E and K196T; P94S/R, D160N, D165A, Y187L, E189R, K190E and K196T; P94S/R, D124G, D160N, D165A, Y187L, E189R, K190E and K196T; D124G, Y187L, E189R and F200L; or P94S/R, D124G, D160N, D165A, Y187L, E189R, K190E, K196T and F200L. In some embodiments, the S. aureus SrtA variants have 59 or 60 (e.g., 25, 30, 35, 40, 45, 50, 55, 59 or 60) amino acids being removed from N-terminus. In some embodiments, the mutated amino acid positions above are numbered according to the numbering of a wild type S. aureus SrtA, e.g., as shown in SEQ ID NO: 1. In some embodiments, the full length nucleotide sequence of the wild type S. aureus SrtA is shown as in e.g., SEQ ID NO: 2.

```
(full length, GenBank Accession No.:
CAA3829591.1)
SEQ ID NO: 1
    1 MKKWTNRLMT IAGVVLILVA AYLFSKPHID
        NYLHDKDKDE KIEQYDKNVK
  51 EQASKDKKQQ AKPQIPKDKS KVAGYIEIPD
        ADIKEPVYPG PATPEQLNRG
101 VSFAEENESL DDQNISIAGH TFIDRPNYQF
        TNLKAAKKGS MVYFKVGNET
151 RKYKMTSIRD VKPTDVGVLD EQKGKDKQLT
        LITCDDYNEK TGVWEKRKIF
201 VATEVK
(full length, wild type)
SEQ ID NO: 2
ATGAAAAAATGGACAAATCGATTAATGACAATCGCT
GGTGTGGTACTTATCCTAGTGGCAGCATATTTGTT
TGCTAAACCACATATCGATAATTATCTTCACGATA
AAGATAAAGATGAAAAGATTGAACAATATGATAAA
AATGTAAAAGAACAGGCGAGTAAAGATAAAAAGCA
GCAAGCTAAACCTCAAATTCCGAAAGATAAATCGA
AAGTGGCAGGCTATATTGAAATTCCAGATGCTGAT
ATTAAAGAACGAGTATATCGAGGACGAGCAACACC
TGAACAATTAAATAGAGGTGTAAGCTTTGCAGAAG
AAAATGAATCACTAGATGATCAAAATATTTCAATT
GCAGGACACACTTTCATTGACCGTCCGAACTATCA
ATTTACAAATCTTAAAGGAGCCAAAAAAGGTAGTA
TGGTGTACTTTAAAGTTGGTAATGAAACACGTAAG
TATAAAATGACAAGTATAAGAGATGTTAAGCCTAC
AGATGTAGGAGTTCTAGATGAACAAAAAGGTAAAG
ATAAACAATTAACATTAATTACTTGTGATGATTAC
AATGAAAAGACAGGCGTTTGGGAAAAACGTAAAAT
CTTTGTAGCTACAGAAGTCAAA
```

In some embodiments, as compared to a wild type S. aureus SrtA, the S. aureus SrtA variant may comprise one or more mutations at one or more of the positions corresponding to 94, 105, 108, 124, 160, 165, 187, 189, 190, 196 and 200 of SEQ ID NO: 1. In some embodiments, as compared to a wild type S. aureus SrtA, the S. aureus SrtA variant may comprise one or more mutations corresponding to P94S/R, E105K, E108A, D124G, D160N, D165A, Y187L, E189R, K190E, K196T and F200L. In some embodiments, as compared to a wild type S. aureus SrtA, the S. aureus SrtA variant may comprise one or more mutations corresponding to D124G, Y187L, E189R and F200L and optionally further comprises one or more mutations corresponding to P94S/R, D160N, D165A, K190E and K196T and optionally further one or more mutations corresponding to E105K and E108A. In certain embodiments, as compared to a wild type S. aureus SrtA, the S. aureus SrtA variant may comprise mutations corresponding to D124G; D124G and F200L; P94S/R, D124G, D160N, D165A, K190E and K196T; P94S/R, D160N, D165A, Y187L, E189R, K190E and K196T; P94S/R, D124G, D160N, D165A, Y187L, E189R, K190E and K196T; D124G, Y187L, E189R and F200L; or P94S/R, D124G, D160N, D165A, Y187L, E189R, K190E, K196T and F200L. In some embodiments, the S. aureus SrtA variant may comprise one or more mutations of P94S/R, E105K, E108A, D124G, D160N, D165A, Y187L, E189R, K190E, K196T and F200L relative to SEQ ID NO: 1. In some embodiments, the S. aureus SrtA variant may comprise D124G, Y187L, E189R and F200L and optionally further comprises one or more mutations of P94S/R, D160N, D165A, K190E and K196T and optionally further comprises E105K and/or E108A relative to SEQ ID NO: 1. In certain embodiments, the S. aureus SrtA variant may, comprise, relative to SEQ ID NO: 1, D124G; D124G and F200L; P94S/R, D124G, D160N, D165A, K190E and K196T; P94S/R, D160N, D165A, Y187L, E189R, K190E and K196T; P94S/R, D124G, D160N, D165A, Y187L, E189R, K190E and K196T; D124G, Y187L, E189R and F200L; or P94S/R, D124G, D160N, D165A, Y187L, E189R, K190E, K196T and F200L. In some embodiments, mutations E105K and/or E108A/Q allows the sortase-mediated reaction to be Ca²⁺ independent. In some embodiments, the S. aureus SrtA variants as described herein may have 25-60 (e.g., 25, 30, 35, 40, 45, 50, 55, 56, 57, 58, 59, or 60) amino acids being removed from N-terminus. In some embodiments, the mutated amino acid positions above are numbered according to the numbering of a full length of a wild type S. aureus SrtA, e.g., as shown in SEQ ID NO: 1.

In some embodiments, a functional variant of S. aureus SrtA useful in the present disclosure may be a S. aureus SrtA variant comprising one or more mutations of P94S/R, E105K, E108A/Q, D124G, D160N, D165A, Y187L, E189R, K190E, K196T and F200L. In certain embodiments, the S. aureus SrtA variant may comprise P94S/R, E105K, E108Q, D124G, D160N, D165A, Y187L, E189R, K190E, K196T and F200L; or P94S/R, E105K, E108A, D124G, D160N, D165A, Y187L, E189R, K190E, K196T and F200L. In some embodiments, the S. aureus SrtA variant may comprise one or more mutations of P94S/R, E105K, E108A/Q, D124G, D160N, D165A, Y187L, E189R, K190E, K196T and F200L relative to SEQ ID NO: 1. In certain embodiments, the S. aureus SrtA variant may comprise P94S/R, E105K, E108Q, D124G, D160N, D165A, Y187L, E189R, K190E, K196T and F200L relative to SEQ ID NO: 1; or P94S/R, E105K, E108A, D124G, D160N, D165A, Y187L, E189R, K190E, K196T and F200L relative to SEQ ID NO: 1. In some embodiments, the S. aureus SrtA variants have 25-60 (e.g., 25, 30, 35, 40, 45, 50, 55, 56, 57, 58, 59, or 60) amino acids being removed from N-terminus. In some embodiments, the mutated amino acid positions above are numbered according to the numbering of a wild type S. aureus SrtA, e.g., as shown in SEQ ID NO: 1.

In some embodiments, the present disclosure contemplates a S. aureus SrtA variant (mg SrtA) comprising or consisting essentially of or consisting of an amino acid sequence having at least 60% (e.g., at least 65%, 70%, 75%, 80%, 85%, 90%, 91%, 92%, 93%, 94%, 95%, 96%, 97%, 98%, 99%, 99.5%, 99.9% or higher) identity to an amino acid sequence as set forth in SEQ ID NO: 3. In some embodiments, SEQ ID NO: 3 is a truncated SrtA and the mutations corresponding to wild type SrtA are shown in bold and underlined below. In some embodiments, the SrtA variant comprises or consists essentially of or consists of an amino acid sequence having at least 60% (e.g., at least 65%, 70%, 75%, 80%, 85%, 90%, 91%, 92%, 93%, 94%, 95%, 96%, 97%, 98%, 99%, 99.5%, 99.9% or higher) identity to an amino acid sequence as set forth in SEQ ID NO: 3 and comprises the mutations of P94R/S, D124G, D160N, D165A, Y187L, E189R, K190E, K196T and F200L and optionally E105K and/or E108A/Q (numbered according to the numbering of SEQ ID NO: 1).

```
(mutations shown in bold and underlined)
SEQ ID NO: 3
    1 KPHIDNYLHD KDKDEKIEQY DKNVKEQASK
        DKKQQAKPQI PKDKSKVAGY
  51 IEIPDADIKE PVYPGPATRE QLNRGVSFAE
        ENESLDDQNI SIAGHTFIGR
101 PNYQFTNLKA AKKGSMVYFK VGNETRKYKM
        TSIRNVKPTA VGVLDEQKGK
151 DKQLTLITCD DLNRETGVWE TRKILVATEV
        K
```

In some embodiments, the present disclosure provides a nucleic acid encoding the S. aureus SrtA variant, and in some embodiments the nucleic acid is set forth in SEQ ID NO: 4.

SEQ ID NO: 4
AAACCACATATCGATAATTATCTTCACGATA
AAGATAAAGATGAAAAGATTGAACAATATGA
TAAAAATGTAAAAGAACAGGCGAGTAAAGAT
AAAAAGCAGCAAGCTAAACCTCAAATTCCGA
AAGATAAATCGAAAGTGGCAGGCTATATTGA
AATTCCAGATGCTGATATTAAAGAACCAGTA
TATCCAGGACCAGCAACACGTGAACAATTAA
ATAGAGGTGTAAGCTTTGCAGAAGAAAATGA
ATCACTAGATGATCAAAATATTTCAATTGCA
GGACACACTTTCATTGGCCGTCCGAACTATC
AATTTACAAATCTTAAAGCAGCCAAAAAAGG
TAGTATGGTGTACTTTAAAGTTGGTAATGAA
ACACGTAAGTATAAAATGACAAGTATAAGAA
ATGTTAAGCCTAGAGCTGTAGGAGTTCTAGA
TGAACAAAAAGGTAAAGATAAACAATTAACA
TTAATTACTTGTGATGATCTTAATCGGGAGA
CAGGCGTTTGGGAAACACGTAAAATCTTGGT
AGCTACAGAAGTCAAA

In some embodiments, the S. aureus SrtA variant can be a truncated variant with e.g. 25-60 (e.g., 30, 35, 40, 45, 50, 55, 59 or 60) amino acids being removed from N-terminus (as compared to the wild type S. aureus SrtA). In some embodiments, the truncated variant comprises or consists essentially of or consists of an amino acid sequence having at least 60% (e.g., at least 65%, 70%, 75%, 80%, 85%, 90%, 91%, 92%, 93%, 94%, 95%, 96%, 97%, 98%, 99%, 99.5%, 99.9% or higher such as 100%) identity to an amino acid sequence as set forth in SEQ ID NO: 27 or 29. The nucleic acids encoding SEQ ID NOs: 28 and 30 are set forth in SEQ ID NOs: 6 and 8 below.

(mutations as compared to wt SrtA
being shown in bold and underlined)
SEQ ID NO: 27
QAKPQI PKDKSKVAGY IEIPDADIKE
PVYPGPATRE QLNRGVSFAE ENESLDDQNI
SIAGHTFIGR PNYQFTNLKA AKKGSMVYFK
VGNETRKYKM TSIRNVKPTA VGVLDEQKGK
DKQLTLITCD DLNRETGVWE TRKILVATEV
K
SEQ ID NO: 28
CAAGCTAAACCTCAAATTCCGAAAGATAAAT
CGAAAGTGGCAGGCTATATTGAAATTCCAGA
TGCTGATATTAAAGAACGAGTATATCGAGGA
GGAGCAACACGTGAACAATTAAATAGAGGTG
TAAGCTTTGCAGAAGAAAATGAATCACTAGA
TGATCAAAATATTTCAATTGCAGGACACACT
TTCATTGGCCGTCCGAACTATCAATTTACAA
ATCTTAAAGCAGCCAAAAAAGGTAGTATGGT
GTACTTTAAAGTTGGTAATGAAACACGTAAG
TATAAAATGACAAGTATAAGAAATGTTAAGC
CTACAGCTGTAGGAGTTCTAGATGAACAAAA
AGGTAAAGATAAACAATTAACATTAATTACT
TGTGATGATCTTAATCGGGAGACAGGCGTTT
GGGAAACACGTAAAATCTTGGTAGCTACAGA
AGTCAAA
(mutations as compared to wt SrtA
being shown in bold and underlined)
SEQ ID NO: 29
MQAKPQI PKDKSKVAGY IEIPDADIKE
PVYPGPATRE QLNRGVSFAE ENESLDDQNI
SIAGHTFIGR PNYQFTNLKA AKKGSMVYFK
VGNETRKYKM TSIRNVKPTA VGVLDEQKGK
DKQLTLITCD DLNRETGVWE TRKILVATEV
K
SEQ ID NO: 30
ATGCAAGCTAAACCTCAAATTCCGAAAGATA
AATCGAAAGTGGCAGGCTATATTGAAATTCC
AGATGCTGATATTAAAGAACCAGTATATCCA
GGACCAGCAACACGTGAACAATTAAATAGAG
GTGTAAGCTTTGCAGAAGAAAATGAATCACT
AGATGATCAAAATATTTCAATTGCAGGACAC
ACTTTCATTGGCCGTCCGAACTATCAATTTA
CAAATCTTAAAGCAGCCAAAAAAGGTAGTAT
GGTGTACTTTAAAGTTGGTAATGAAACACGT
AAGTATAAAATGACAAGTATAAGAAATGTTA
AGCCTACAGCTGTAGGAGTTCTAGATGAACA
AAAAGGTAAAGATAAACAATTAACATTAATT
ACTTGTGATGATCTTAATCGGGAGACAGGCG
TTTGGGAAACACGTAAAATCTTGGTAGCTAC
AGAAGTCAAA

In some embodiments, a sortase A variant may comprise any one or more of the following: an S residue at position 94 (S94) or an R residue at position 94 (R94), a K residue at position 105 (K105), an A residue at position 108 (A108) or a Q residue at position 108 (Q 108), a G residue at position 124 (G124), an N residue at position 160 (N160), an A residue at position 165 (A165), a R residue at position 189 (R189), an E residue at position 190 (E190), a T residue at position 196 (T196), and an L residue at position 200 (L200) (numbered according to the numbering of a wild type SrtA, e.g., SEQ ID NO: 1), optionally with about 25-60 (e.g., 25, 30, 35, 40, 45, 50, 55, 56, 57, 58, 59, or 60) amino acids being removed from N-terminus of the wild type S. aureus SrtA. For example, in some embodiments a sortase A variant comprises two, three, four, or five of the afore-mentioned mutations relative to a wild type S. aureus SrtA (e.g., SEQ ID NO: 1). In some embodiments a sortase A variant comprises an S residue at position 94 (S94) or an R residue at position 94 (R94), and also an N residue at position 160 (N160), an A residue at position 165 (A165), and a T residue at position 196 (T196) relative to a wild type S. aureus SrtA (e.g., SEQ ID NO: 1). For example, in some embodiments, a sortase A variant comprises P94S or P94R, and also D160N, D165A, and K196T relative to a wild type S. aureus SrtA (e.g., SEQ ID NO: 1). In some embodiments a sortase A variant comprises an S residue at position 94 (S94) or an R residue at position 94 (R94) and also an N residue at position 160 (N160), A residue at position 165 (A165), an E residue at position 190, and a T residue at position 196 relative to a wild type S. aureus SrtA (e.g., SEQ ID NO: 1). For example, in some embodiments a sortase A variant comprises P94S or P94R, and also D160N, D165A, K190E, and K196T relative to a wild type S. aureus SrtA (e.g., SEQ ID NO: 1). In some embodiments a sortase A variant comprises an R residue at position 94 (R94), an N residue at position 160 (N160), a A residue at position 165 (A165), E residue at position 190, and a T residue at position 196 relative to a wild type S. aureus SrtA (e.g., SEQ ID NO: 1). In some embodiments a sortase comprises P94R, D160N, D165A, K190E, and K196T relative to a wild type S. aureus SrtA (e.g., SEQ ID NO: 1). In some embodiments, the S. aureus SrtA variants may have 25-60 (e.g., 25, 30, 35, 40, 45, 50, 55, 56, 57, 58, 59 or 60) amino acids being removed from N-terminus.

In some embodiments, a sortase A variety having higher transamidase activity than a naturally occurring sortase A may be used. In some embodiments the activity of the sortase A variety is at least about 10, 15, 20, 40, 60, 80, 100, 120, 140, 160, 180, or 200 times as high as that of wild type S. aureus sortase A. In some embodiments such a sortase variant is used in a composition or method of the present disclosure. In some embodiments a sortase variant comprises any one or more of the following substitutions relative to a wild type S. aureus SrtA: P94S/R, E105K, E108A, E108Q, D124G, D160N, D165A, Y187L, E189R, K190E, K196T and F200L mutations. In some embodiments, the SrtA variant may have 25-60 (e.g., 30, 35, 40, 45, 50, 55, 59 or 60) amino acids being removed from N-terminus.

In some embodiments, the amino acid mutation positions are determined by an alignment of a parent S. aureus SrtA (from which the S. aureus SrtA variant as described herein is derived) with the polypeptide of SEQ ID NO: 1, i.e., the polypeptide of SEQ ID NO: 1 is used to determine the corresponding amino acid sequence in the parent S. aureus SrtA. Methods for determining an amino acid position corresponding to a mutation position as described herein is well known in the art. Identification of the corresponding amino acid residue in another polypeptide can be confirmed by using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. Based on above well-known computer programs, it is routine work for those of skills to determine the amino acid position of a polypeptide of interest as described herein.

In some embodiments, the sortase variant may further comprise 1, 2, 3, 4, 5, 6, 7, 8, 9, 10, 11, 12, 13, 14, or 15 conservative amino acid mutations. Conservative amino acid mutations that will not substantially affect the activity of a protein are well known in the art.

In some embodiments, the present disclosure provides a method of identifying a sortase variant candidate for conjugating an agent to at least one endogenous, non-engineered membrane protein of a red blood cell, comprising contacting the red blood cell with a sortase substrate that comprises a sortase recognition motif and an agent, in the presence of the sortase variant candidate under conditions suitable for the sortase variant candidate to conjugate the sortase substrate to the at least one endogenous, non-engineered membrane protein of the RBC by a sortase-mediated reaction, preferably by a sortase-mediated glycine conjugation and/or a sortase-mediated lysine side chain ε-amino group conjugation. In some embodiments, the sortase-mediated glycine conjugation and/or the sortase-mediated lysine side chain ε-amino group conjugation occur at least on glycine₍ₙ₎ and/or lysine ε-amino group at internal sites of the extracellular domain of the at least one endogenous, non-engineered membrane protein, preferably n being 1 or 2. In some embodiments, the method further comprises selecting the sortase variant capable of conjugating an agent to at least one endogenous, non-engineered membrane protein of a red blood cell.

In some embodiments, the present disclosure contemplates administering a sortase and a sortase substrate to a subject to conjugate in vivo the sortase substrate to red blood cells. For this purpose, it is desirable to use a sortase that has been further modified to enhance its stabilization in circulation and/or reduce its immunogenicity. Methods for stabilizing an enzyme in circulation and for reducing enzyme immunogenicity are well known in the art. For example, in some embodiments, the sortase has been PEGylated and/or linked to an Fc fragment at a position that will not substantially affect the activity of the sortase.

### Sortase Substrates

Substrates suitable for a sortase-mediated conjugation can readily be designed. A sortase substrate may comprises a sortase recognition motif and an agent. For example, an agent such as polypeptides can be modified to include a sortase recognition motif at or near their C-terminus, thereby allowing them to serve as substrates for sortase. The sortase recognition motif need not be positioned at the very C-terminus of a substrate but should typically be sufficiently accessible by the enzyme to participate in the sortase reaction. In some embodiments a sortase recognition motif is considered to be “near” a C-terminus if there are no more than 5, 6, 7, 8, 9, 10 amino acids between the most N-terminal amino acid in the sortase recognition motif (e.g., L) and the C-terminal amino acid of the polypeptide. A polypeptide comprising a sortase recognition motif may be modified by incorporating or attaching any of a wide variety of moieties (e.g., peptides, proteins, compounds, nucleic acids, lipids, small molecules and sugars) thereto.

### Agents

Depending on the intended applications of the modified red blood cells, a wide variety of agents such as a binding agent, a therapeutic agent or a detection agent can be contemplated in the present disclosure. In some embodiments, an agent may comprise a protein, a peptide (e.g., an extracellular domain of oligomeric ACE2), an antibody or its functional antibody fragment, an antigen or epitope, a MHC-peptide complex, a drug such as a small molecule drug (e.g., an antitumor agent such as a chemotherapeutic agent), an enzyme (e.g., a functional metabolic or therapeutic enzyme), a hormone, a cytokine, a growth factor, an antimicrobial agent, a probe, a ligand, a receptor, an immunotolerance-inducing peptide, a targeting moiety or any combination thereof.

In some embodiments, in addition to a therapeutically active domain such as an enzyme, a drug, a small molecule (such as a small molecule drug (e.g., an antitumor agent such as a chemotherapeutic agent)), a therapeutic protein and a therapeutic antibody as described herein, the agent may further comprise a targeting moiety for targeting the cells and/or agent to a site in the body where the therapeutic activity is desired. The targeting moiety binds to a target present at such a site. Any targeting moiety may be used, e.g., an antibody. The site may be any organ or tissue, e.g., respiratory tract (e.g., lung), bone, kidney, liver, pancreas, skin, cardiovascular system (e.g., heart), smooth or skeletal muscle, gastrointestinal tract, eye, blood vessel surfaces, etc.

In some embodiments, a protein is an enzyme such as a functional metabolic or therapeutic enzyme, e.g., an enzyme that plays a role in metabolism or other physiological processes in a mammal. In some embodiments a protein is an enzyme that plays a role in carbohydrate metabolism, amino acid metabolism, organic acid metabolism, porphyrin metabolism, purine and/or pyrimidine metabolism. Deficiencies of enzymes or other proteins can lead to a variety of diseases, e.g., diseases associated with defects in carbohydrate metabolism, amino acid metabolism, organic acid metabolism, purine or pyrimidine metabolism, and blood clotting, among others. Metabolic diseases are characterized by the lack of functional enzymes or excessive intake of metabolites. Thus, the metabolites deposition in the circulation and tissues causes tissue damage. Due to the wide distribution in human body of RBCs, the present disclosure contemplates modifying membrane proteins of RBCs with functional metabolic enzymes. The enzymes targeted RBCs will uptake metabolites in plasma of patients. Exemplary enzymes include acetaldehyde dehydrogenase for alcoholic hepatitis, butyrylcholinesterase for cocaine metabolite, and the like.

In some embodiments, the agent may comprise a peptide. Various functional peptides can be contemplated in the present disclosure. In certain embodiment, the peptide may comprise an oligomeric ACE2 extracellular domain.

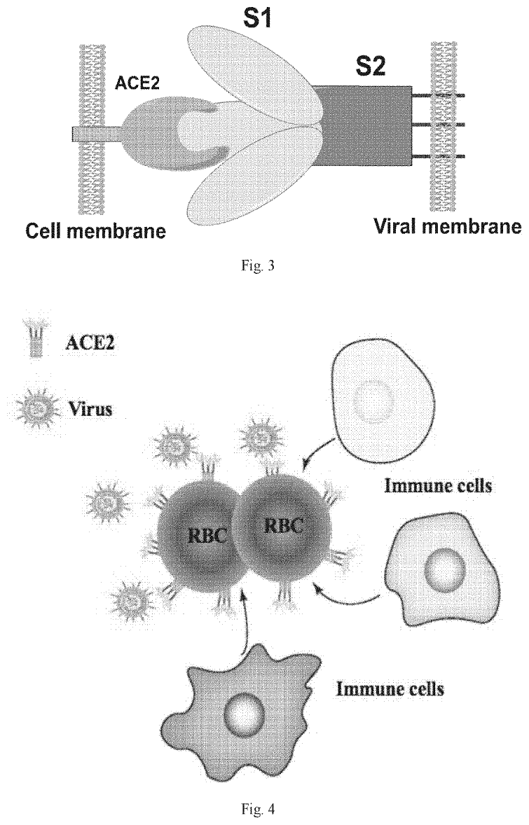

SARS-CoV-2, which causes a respiratory disease named COVID-19, belongs to the same coronaviridea as SARS-CoV. The genome of SARS-CoV-2 is very similar to SARS-CoV sharing ˜80% nucleotide sequence identity and 94.6% amino acid sequence identity in the ORF encoding the spike protein. SARS-CoV-2 and SARS-CoV spike proteins have very similar structures, both entering human cells through spike protein interaction with ACE2 as shown in FIG. 3. Unfortunately, seventeen years after SARS pandemic, no effective detection (except RT-PCR), prevention or treatment approaches were developed from SARS-CoV that could be readily applied to SARS-CoV-2. This has caught everybody in a hurry to come up with different strategies including SARS-CoV-2 specific antibodies, vaccines, protease inhibitors and RNA-dependent RNA polymerase inhibitors to detect and combat SARS-CoV-2 infected disease “COVID-19”. These efforts may be useful for SARS-CoV-2 if developed quick enough (probably within 2-3 months). However, they still may not be applied to future coronavirus given the fact that RNA viruses have a really high mutation rate. The lack of cross-reactivity between several SARS-CoV specific antibodies and SARS-CoV-2 is a clear demonstration for this. Thus, detection devices or therapeutic agents which are not only useful for SARS-CoV-2, but also could be readily applied to future coronavirus are highly desirable for development.

Both SARS-CoV and SARS-CoV-2 enter host cells through binding with ACE2 by its S protein. This mechanism is also applying to other coronavirus in order to successfully establish the infection. Thus, molecules blocking S protein interaction with ACE2 could prevent virus infection. It has been shown ACE2 extracellular domain could block virus infection. However, monomeric ACE2 only has limited binding affinity to S protein and is not expected to have a high virus blocking activity. High-affinity oligomeric ACE2 on the other hand possess a high virus binding affinity and could effectively compete with cell surface ACE2 for virus neutralization.

Cell assays have demonstrated coronavirus infection or even S protein binding with ACE2 will cause shedding of ACE2 from cell surface, resulting in decreased cell surface ACE2 expression level[11][12]. Down regulation of ACE2 results in angiotensin II accumulation which is closely related with acute lung injury[11][13][14]. This perhaps could explain the fact that coronavirus infected patients show respiratory syndromes especially in the lung. The fact that coronavirus infected patients show respiratory syndromes and some even develop ARDS suggests supplementing ACE2 could also alleviate respiratory syndromes for virus infection treatment.

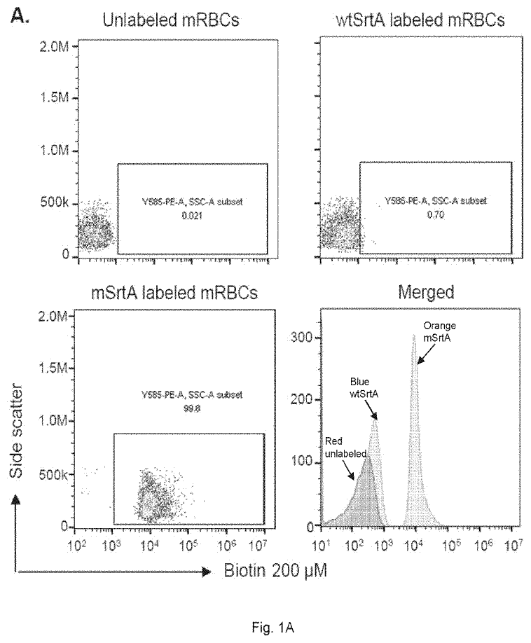

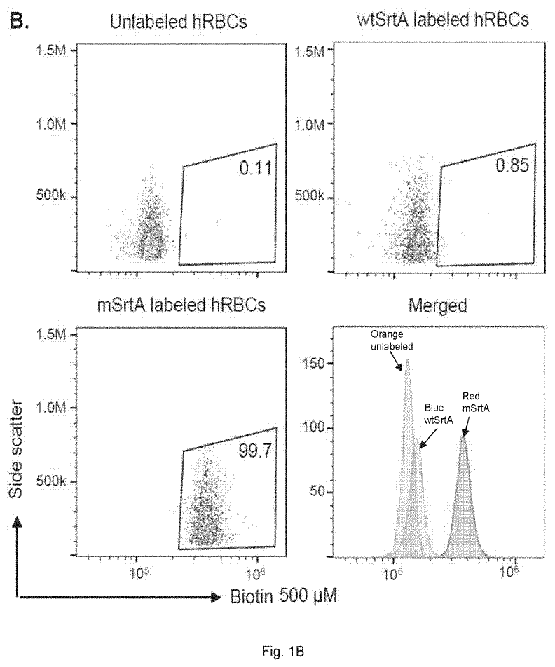

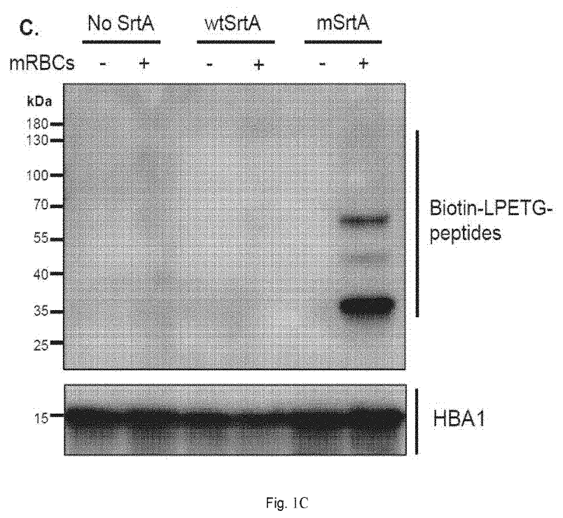

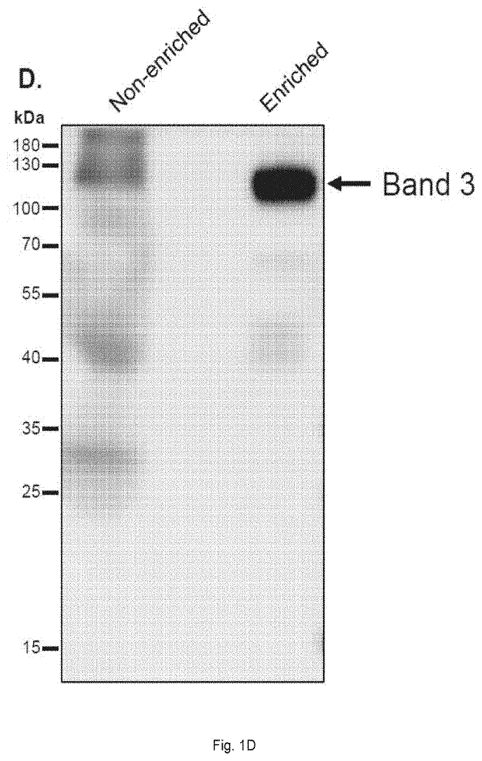

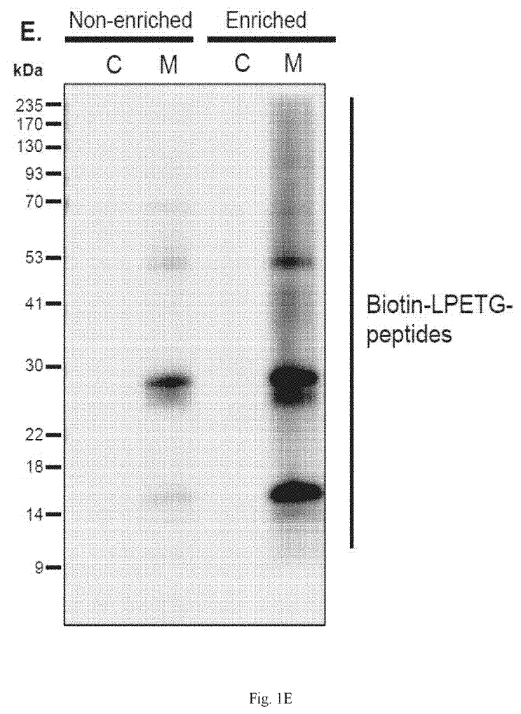

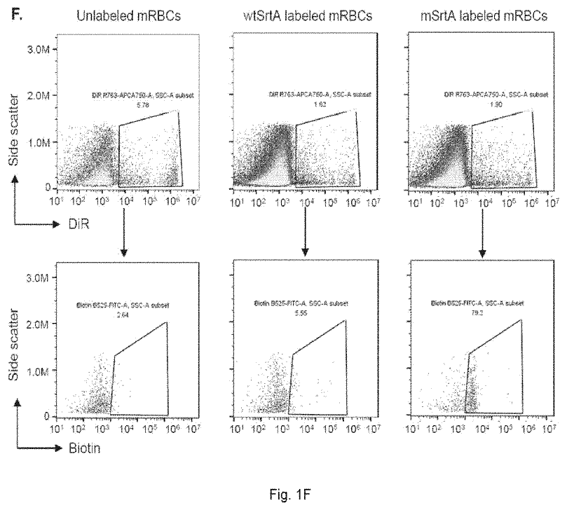

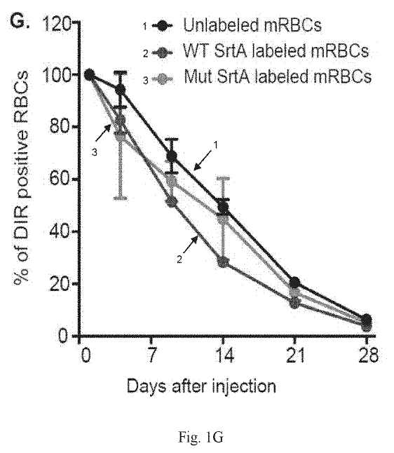

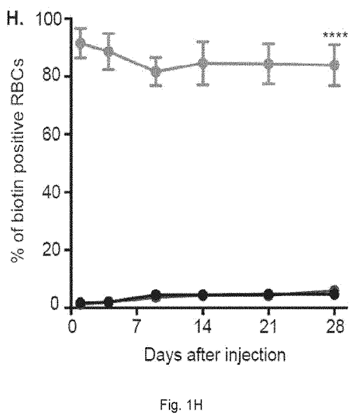

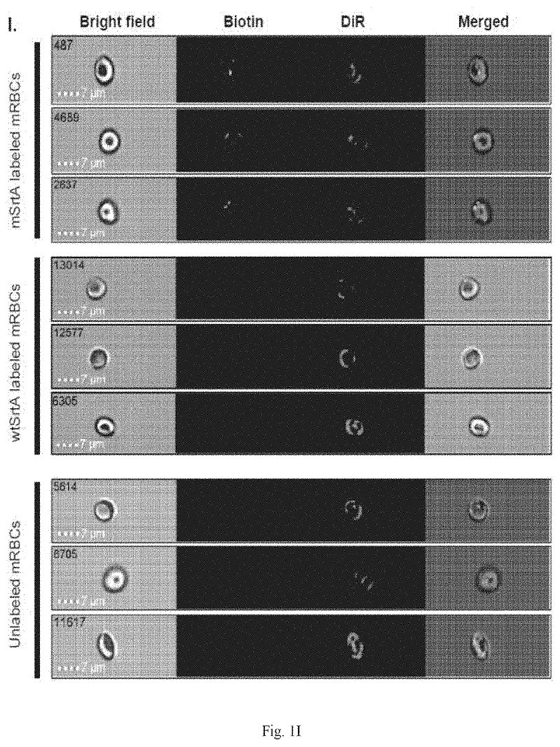

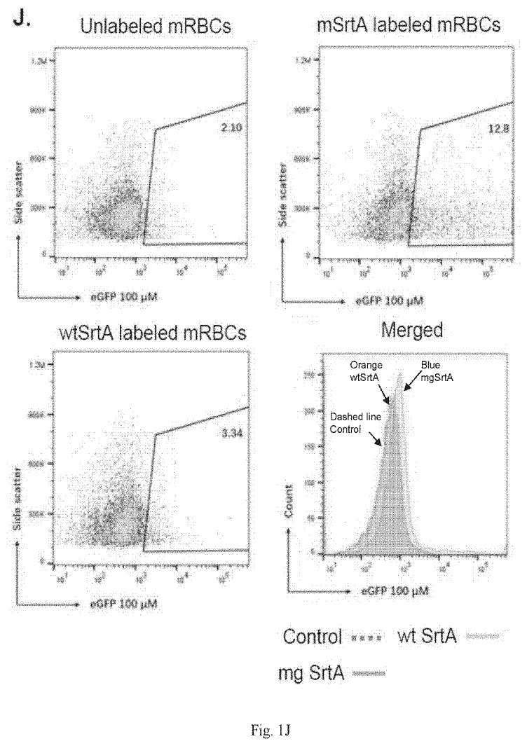

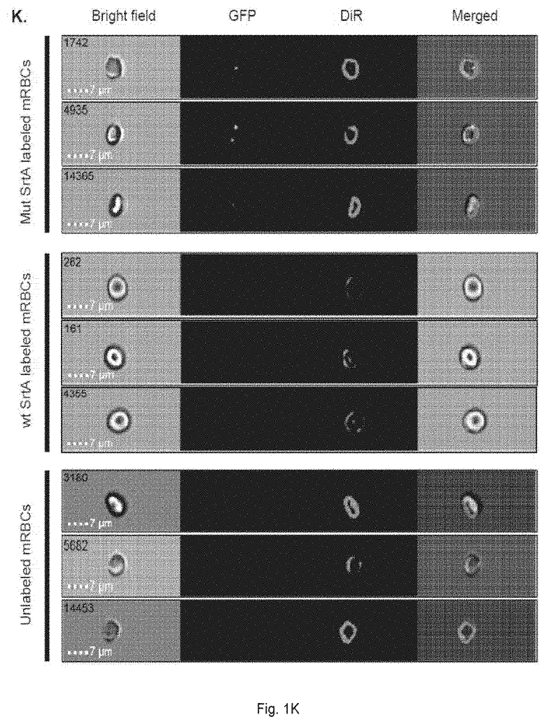

In some embodiments, the present disclosure contemplates using red blood cells as oligomeric ACE2 carrier for effective virus neutralization (FIG. 4), by use of the new strategy to covalently modify endogenous membrane proteins of natural RBCs with peptides and/or small molecules through an mg SrtA-mediated reaction as described herein. In the present disclosure, the inventors have already characterized the efficacy of mg SrtA-mediated protein labeling on RBC membranes in vivo. GFP labeled mouse RBCs, which were simultaneously labeled with a fluorescent dye DiR (1,1′-dioctadecyl-3,3,3′,3′-tetramethylindotricarbocyanine iodide), were transfused into wildtype recipient mice. The percentage of DiR and GFP positive RBCs in vivo was analyzed periodically. It was found that GFP tagged RBCs not only showed the same lifespan as the control groups, but also remained 90% GFP positive during circulation (FIGS. 1G and 1F). Imaging analysis also showed convincing GFP signals on the cell surface and normal morphology of engineered RBCs (FIG. 1K). Taken together, the data suggests efficient labeling proteins on the surface of natural RBCs mediated by sortase enzyme. Based on these data, it is believed that high-affinity oligomeric ACE2 linked to red blood cells by the covalently modifying method of the present disclosure could not only neutralize virus particles, but also supplement the lost cell surface ACE2 to alleviate lung injury and thus be used for current and future coronavirus infection prevention and treatment.

In some embodiments, the agent may comprise an antibody, including an antibody, an antibody chain, an antibody fragment e.g., scFv, an antigen-binding antibody domain, a VHH domain, a single-domain antibody, a camelid antibody, a nanobody, an adnectin, or an anticalin. The red blood cells having antibodies attached thereto may be used as a delivery vehicle for the antibodies and/or the antibodies may serve as a targeting moiety. Exemplary antibodies include anti-tumor antibodies. The heavy chains of the antibodies modified with a sortase recognition motif such as LPETG can be expressed and purified. Adalimumab, Infliximab, Sarilumab and Golimumab which are FDA approved therapeutic monoclonal antibodies for curing rheumatoid arthritis can be modified by using the method as described herein.

In some embodiments, the agent may comprise an antigen or epitopes or a binding moiety that binds to an antigen or epitope. In some embodiments an antigen is any molecule or complex comprising at least one epitope recognized by a B cell and/or by a T cell. An antigen may comprise a polypeptide, a polysaccharide, a carbohydrate, a lipid, a nucleic acid, or combination thereof. An antigen may be naturally occurring or synthetic, e.g., an antigen naturally produced by and/or is genetically encoded by a pathogen, an infected cell, a neoplastic cell (e.g., a tumor or cancer cell), a virus, bacteria, fungus, or parasite. In some embodiments, an antigen is an autoantigen or a graft-associated antigen. In some embodiments, an antigen is an envelope protein, capsid protein, secreted protein, structural protein, cell wall protein or polysaccharide, capsule protein or polysaccharide, or enzyme. In some embodiments an antigen is a toxin, e.g., a bacterial toxin. An antigen or epitope may be modified, e.g., by conjugation to another molecule or entity (e.g., an adjuvant).

In some embodiments, red blood cells having an epitope, antigen or portion thereof conjugated thereto by sortase as described herein may be used as vaccine components. In some embodiments an antigen conjugated to red blood cells using sortase as described herein may be any antigen used in a conventional vaccine known in the art.

In some embodiments an antigen is a surface protein or polysaccharide of, e.g., a viral capsid, envelope, or coat, or bacterial, fungal, protozoal, or parasite cell. Exemplary viruses may include, e.g., coronaviruses (e.g., SARS-CoV and SARS-CoV-2), HIV, dengue viruses, encephalitis viruses, yellow fever viruses, hepatitis virus, Ebola viruses, influenza viruses, and herpes simplex virus (HSV) 1 and 2.

In some embodiments an antigen is a tumor antigen (TA), which can be any antigenic substance produced by cells in a tumor, e.g., tumor cells or in some embodiments tumor stromal cells (e.g., tumor-associated cells such as cancer-associated fibroblasts or tumor-associated vasculature).

In some embodiments, an antigen is a peptide. Peptides may bind directly to MHC molecules expressed on cell surfaces, may be ingested and processed by APC and displayed on APC cell surfaces in association with MHC molecules, and/or may bind to purified MHC proteins (e.g., WIC oligomers). In some embodiments a peptide contains at least one epitope capable of binding to an appropriate WIC class I protein and/or at least one epitope capable of binding to an appropriate MHC class II protein. In some embodiments a peptide comprises a CTL epitope (e.g., the peptide can be recognized by CTLs when bound to an appropriate WIC class I protein).

In some embodiments, the agent may comprise a WIC-peptide complex, which may comprise a WIC and a peptide such as an antigenic peptide or an antigen as described herein for activating immune cells. In some embodiments, the antigenic peptide is associated with a disorder and is able to activate CD8⁺ T cells when presented by a WIC class I molecule. Class-I major histocompatibility complex (WIC-I) is presenting antigen peptides to and activating immune cells particularly CD8⁺ T cells, which are important for fighting against cancers, infectious diseases, etc. WIC-peptide complexes with sortase recognition motifs such as LPETG can be expressed and purified exogenously through eukaryotic or prokaryotic systems. The purified MHC-peptide complexes will be covalently bound to RBCs by sortase-mediated reactions as described herein. In the present disclosure, we used MHC-I-OT1 complex as an example. Mouse MHC-I-OT1 protein is expressed by E. coli and purified by histidine-tagged affinity chromatography. The purified MHC-I-OT1 complexes are successfully ligated on membrane proteins of RBCs. Similarly, MHC-II is presenting antigen peptides to and activating immune cells particularly CD4⁺ T cells and thus a MHC complex comprising MHC-II and an antigen or an antigenic peptide can be covalently bound to RBCs by sortase-mediated reactions as described herein.

This strategy of MHC complex can be used to treat or prevent diseases caused by viruses, such as HPV (targeting E6/E7), coronavirus (e.g., targeting SARS-CoV or SARS-CoV-2 Spike protein), and influenza virus (e.g., targeting H antigen/N antigen). This strategy of WIC complex can also be used to target tumor mutations, for example Kras with mutations such as V8M and/or G12D, Alk with a mutation such as E1171D, Braf with a mutation such as W487C, Jak2 with a mutation such as E92K, Stat3 with a mutation such as M28I, Trp53 with mutations such as G242V and/or S258I, Pdgfra with a mutation such as V88I, and Brca2 with a mutation such as R2066K, for tumor treatment.

In some embodiments, the agent may comprise a growth factor. In some embodiments, the agent may comprise a growth factor for one or more cell types. Growth factors include, e.g., members of the vascular endothelial growth factor (VEGF, e.g., VEGF-A, VEGF-B, VEGF-C, VEGF-D), epidermal growth factor (EGF), insulin-like growth factor (IGF; IGF-1, IGF-2), fibroblast growth factor (FGF, e.g., FGF1-FGF22), platelet derived growth factor (PDGF), or nerve growth factor (NGF) families.

In some embodiments, the agent may comprise a cytokine or the biologically active portion thereof. In some embodiments a cytokine is an interleukin (IL) e.g., any of IL-1 to IL-38 (e.g., IL-2, IL-3, IL-4, IL-5, IL-6, IL-7, IL-12), interferons (e.g., a type I interferon, e.g., IFN-α), and colony stimulating factors (e.g., G-CSF, GM-CSF, M-CSF). Cytokine (such as recombinant IL-2, recombinant IL-7, recombinant IL-12) loaded RBCs is a therapeutic delivery system for increasing tumor cytotoxicity and IFN-γ production.

In some embodiments, the agent may comprise a small molecule, e.g., those used as targeting moieties, immunomodulators, detection agents, therapeutic agents, or ligands (such as CD19, CD47, TRAIL, TGF, CD44) to activate or inhibit a corresponding receptor.

In some embodiments, the agent may comprise a receptor or receptor fragment. In some embodiments, the receptor is a cytokine receptor, growth factor receptor, interleukin receptor, or chemokine receptor. In some embodiments a growth factor receptor is a TNFα receptor (e.g., Type I TNF-α receptor), VEGF receptor, EGF receptor, PDGF receptor, IGF receptor, NGF receptor, or FGF receptor. In some embodiments a receptor is TNF receptor, LDL receptor, TGF receptor, or ACE2.

In some embodiments, an agent to be conjugated to red blood cells may comprise an anti-cancer or anti-tumor agent, for example, a chemotherapy drug. In certain embodiments, red blood cells are conjugated both with an anti-tumor agent and a targeting moiety, wherein the targeting moiety targets the red blood cell to a cancer. Anti-cancer agents are conventionally classified in one of the following group: radioisotopes (e.g., Iodine-131, Lutetium-177, Rhenium-188, Yttrium-90), toxins (e.g., diphtheria, Pseudomonas, ricin, gelonin), enzymes, enzymes to activate prodrugs, radio-sensitizing drugs, interfering RNAs, superantigens, anti-angiogenic agents, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens. In some embodiments an anti-tumor agent is a protein such as a monoclonal antibody or a bispecific antibody such as anti-receptor tyrosine kinases (e.g., cetuximab, panitumumab, trastuzumab), anti-CD20 (e.g., rituximab and tositumomab) and others for example alemtuzumab, aevacizumab, and gemtuzumab; an enzyme such as asparaginase; a chemotherapy drug including, e.g., alkylating and alkylating-like agents such as nitrogen mustards; platinum agents (e.g., alkylating-like agents such as carboplatin, cisplatin), busulfan, dacarbazine, procarbazine, temozolomide, thioTEPA, treosulfan, and uramustine; purines such as cladribine, clofarabine, fludarabine, mercaptopurine, pentostatin, thioguanine; pyrimidines such as capecitabine, cytarabine, fluorouracil, floxuridine, gemcitabine; cytotoxic/anti-tumor antibiotics such anthracyclines (e.g., daunorubicin, doxorubicin, epirubicin, idarubicin, mitoxantrone, pixantrone, and valrubicin); and others for example taxol, nocodazole, or β-Ionone. Antitumor agent loaded RBCs via membrane proteins is promising for decreasing antibiotic toxicity and increasing circulation times and can perform as a slow drug delivery.

In some embodiments, a tumor is a malignant tumor or a “cancer”. The term “tumor” includes malignant solid tumors (e.g., carcinomas, sarcomas) and malignant growths with no detectable solid tumor mass (e.g., certain hematologic malignancies). The term “cancer” is generally used interchangeably with “tumor” herein and/or to refer to a disease characterized by one or more tumors, e.g., one or more malignant or potentially malignant tumors. Cancer includes, but is not limited to: breast cancer; biliary tract cancer; bladder cancer; choriocarcinoma; colon cancer; endometrial cancer; esophageal cancer; gastric cancer; hematological neoplasms; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms; liver cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma, pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer; testicular cancer; thyroid cancer.

In some embodiments, an agent to be conjugated to red blood cells may comprise an anti-microbial agent. An anti-microbial agent may include compounds that inhibit proliferation or activity of, destroy or kill bacteria, viruses, fungi, parasites. In some embodiments the red blood cells are conjugated with an anti-microbial agent against a bacteria, virus, fungi, or parasite and with a targeting moiety, wherein the targeting moiety targets the cell to the bacteria, virus, fungi, or parasite. In some embodiments, the anti-microbial agent may include β-lactamase inhibitory proteins or metallo-beta-lactamase for treating bacterial infections.

In some embodiments, an agent to be conjugated to red blood cells may comprise probes, which can be used as for example diagnostic tools. Molecular imaging has been demonstrated as an efficient way for tracking disease progression such as in cancer. Small molecular probes such as fluorescein can be labeled on RBCs through an enzymatic reaction by sortase A as described herein, instead of conventional chemical reaction which may cause damage to cells.

In some embodiments, an agent to be conjugated to red blood cells may comprise a prodrug. The term “prodrug” refers to a compound that, after in vivo administration, is metabolized or otherwise converted to the biologically, pharmaceutically or therapeutically active form of the compound. A prodrug may be designed to alter the metabolic stability or the transport characteristics of a compound, to mask side effects or toxicity, to improve the flavor of a compound and/or to alter other characteristics or properties of a compound. By virtue of knowledge of pharmacodynamic processes and drug metabolisms in vivo, once a pharmaceutically active compound is identified, those of skills in the pharmaceutical art generally can design prodrugs of the compound (Nogrady, “Medicinal Chemistry A Biochemical Approach”, 1985, Oxford University Press: N.Y., pages 388-392). Procedures for the selection and preparation of suitable prodrugs are also known in the art. In the context of the present invention, a prodrug is preferably a compound that, after in vivo administration, whose conversion to its active form involves enzymatic catalysis.

### Methods for Covalently Modifying Endogenous, Non-Engineered Membrane Proteins of RBCs

In an aspect, the present disclosure provides a method for covalently modifying at least one endogenous, non-engineered membrane protein of a red blood cell, comprising contacting the RBC with a sortase substrate that comprises a sortase recognition motif and an agent, in the presence of a sortase under conditions suitable for the sortase to conjugate the sortase substrate to the at least one endogenous, non-engineered membrane protein of the RBC by a sortase-mediated reaction, preferably by a sortase-mediated glycine conjugation and/or a sortase-mediated lysine side chain conjugation. In some embodiments, the sortase-mediated glycine conjugation and/or the sortase-mediated lysine side chain ε-amino group conjugation occur at least on glycine₍ₙ₎ and/or lysine ε-amino group in the extracellular domain (for example at internal sites of the extracellular domain) of the at least one endogenous, non-engineered membrane protein, preferably n being 1 or 2. In some embodiments, without being limited to the theory, the sortase-mediated glycine conjugation may also occur at exposed glycine_{(n=1 or 2)} of previously unreported membrane proteins due to tissue-specific mRNA splicing and protein translation during erythropoiesis. In some embodiments, the sortase-mediated lysine side chain ε-amino group conjugation occur at ε-amino group of terminal lysine or internal lysine of the extracellular domain.

It would be understood that those of ordinary skills are able to select conditions (e.g., optimal temperature, pH) suitable for the sortase to conjugate the sortase substrate to the at least one endogenous, non-engineered membrane protein according to the nature of sortase substrate, the type of sortase and the like.

### Uses

Sortagged red blood cells described herein have a number of uses. In some embodiments, the sortagged red blood cells may be used as a vaccine component, a delivery system or a diagnostic tool. In some embodiments, the sortagged red blood cells may be used to treat or prevent various disorders, conditions or diseases as described herein such as tumors or cancers, metabolic diseases, bacterial infections, virus infections such as coronavirus for example SARS-COV or SARS-COV-2 infection, autoimmune diseases or inflammatory diseases. In some embodiments, sortagged red blood cells may be used in cell therapy. In some embodiments cell therapy is administered for treatment of cancer, infections such as bacterial or virus infections, autoimmune diseases, or enzyme deficiencies. In some embodiments, red blood cells sortagged with peptides for inducing immunotolerances may be used to modulate immune response such as inducing immunotolerance. In some embodiments administered red blood cells may originate from the individual to whom they are administered (autologous), may originate from different genetically identical individual(s) of the same species (isogeneic), may originate from different non-genetically identical individual(s) of the same species (allogeneic), or may originate from individual(s) of a different species. In certain embodiments, allogeneic red blood cells may originate from an individual who is immunocompatible with the subject to whom the cells are administered.

In some embodiments, the sortagged red blood cells are used as a delivery vehicle or system for the agent. For example, the sortagged red blood cells that have a protein conjugated to their surface may serve as delivery vehicles for the protein. Such cells may be administered to a subject suffering from a deficiency of the protein or who may benefit from increased levels of the protein. In some embodiments the cells are administered to the circulatory system, e.g., by infusion. Examples of various diseases associated with deficiency of various proteins, e.g., enzymes, are provided above. In some embodiments, using sortagged RBCs as a delivery system can achieve a retention release, for example for delivering hormones like glucocorticoids, insulin and/or growth hormones in a retention release profile.

In some embodiments, the present disclosure provides a method for diagnosing, treating or preventing a disorder, condition or disease in a subject in need thereof, comprising administering the red blood cell or composition as described herein to the subject. In some embodiments, the disorder, condition or disease is selected from a group consisting of tumors or cancers, metabolic diseases, bacterial infections, virus infections such as coronavirus for example SARS-COV or SARS-COV-2 infection, autoimmune diseases and inflammatory diseases.

As used herein, “treating”, “treat” or “treatment” refers to a therapeutic intervention that at least partly ameliorates, eliminates or reduces a symptom or pathological sign of a pathogen-associated disease, disorder or condition after it has begun to develop. Treatment need not be absolute to be beneficial to the subject. The beneficial effect can be determined using any methods or standards known to the ordinarily skilled artisan.

As used herein, “preventing”, “prevent” or “prevention” refers to a course of action initiated prior to infection by, or exposure to, a pathogen or molecular components thereof and/or before the onset of a symptom or pathological sign of the disease, disorder or condition, so as to prevent infection and/or reduce the symptom or pathological sign. It is to be understood that such preventing need not be absolute to be beneficial to a subject. A “prophylactic” treatment is a treatment administered to a subject who does not exhibit signs of the disease, disorder or condition, or exhibits only early signs for the purpose of decreasing the risk of developing a symptom or pathological sign of the disease, disorder or condition.

In some embodiments, the method as described herein further comprises administering the conjugated red blood cells to a subject, e.g., directly into the circulatory system, e.g., intravenously, by injection or infusion.

In another aspect, provided is a method of delivering an agent to a subject in need thereof, comprising administering the red blood cell or the composition as described herein to the subject. The term “delivery” or “delivering” refers to transportation of a molecule or agent to a desired cell or tissue site. Delivery can be to the cell surface, cell membrane, cell endosome, within the cell membrane, nucleus or within the nucleus, or any other desired area of the cell.

In another aspect, provided is a method of increasing the circulation time or plasma half-life of an agent in a subject, comprising providing a sortase substrate that comprises a sortase recognition motif and an agent, and conjugating the sortase substrate in the presence of a sortase under conditions suitable for the sortase to conjugate the sortase substrate to the at least one endogenous, non-engineered membrane protein of a red blood cell by a sortase-mediated reaction, preferably by a sortase-mediated glycine conjugation and/or a sortase-mediated lysine side chain ε-amino group conjugation. In some embodiments the method further comprises administering the red blood cell to the subject, e.g., directly into the circulatory system, e.g., intravenously or by injection or infusion.

In some embodiments, a subject receives a single dose of cells, or receives multiple doses of cells, e.g., between 2 and 5, 10, 20, or more doses, over a course of treatment. In some embodiments a dose or total cell number may be expressed as cells/kg. For example, a dose may be about 10³, 10⁴, 10⁵, 10⁶, 10⁷, 10⁸ cells/kg. In some embodiments a course of treatment lasts for about 1 week to 12 months or more e.g., 1, 2, 3 or 4 weeks or 2, 3, 4, 5 or 6 months. In some embodiments a subject may be treated about every 2-4 weeks. One of ordinary skills in the art will appreciate that the number of cells, doses, and/or dosing interval may be selected based on various factors such as the weight, and/or blood volume of the subject, the condition being treated, response of the subject, etc. The exact number of cells required may vary from subject to subject, depending on factors such as the species, age, weight, sex, and general condition of the subject, the severity of the disease or disorder, the particular cell(s), the identity and activity of agent(s) conjugated to the cells, mode of administration, concurrent therapies, and the like.

### Composition

In another aspect, the present disclosure provides a composition comprising the red blood cell as described herein and optionally a physiologically acceptable carrier, such as in the form of a pharmaceutical composition, a delivery composition or a diagnostic composition or a kit.

In some embodiments, the composition may comprise a plurality of red blood cells. In some embodiments, at least a selected percentage of the cells in the composition are modified, i.e., having an agent conjugated thereto by sortase. For example, in some embodiments at least 5%, 10%, 15%, 20%, 25%, 30%, 40%, 50%, 60%, 70%, 80%, 90%, 95%, 96%, 97%, 98%, 99%, or more of the cells have an agent conjugated thereto. In some embodiments, two or more red blood cells or red blood cell populations conjugated with different agents are included.

In some embodiments, a composition comprises sortagged blood red cells, wherein the cells are sortagged with any agent of interest. In some embodiments, a composition comprises an effective amount of cells, e.g., up to about 10¹⁴ cells, e.g., about 10, 10², 10³, 10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², 5×10¹² 10¹³, 5×10¹³ or 10¹⁴ cells. In some embodiments the number of cells may range between any two of the afore-mentioned numbers.

As used herein, the term “an effective amount” refers to an amount sufficient to achieve a biological response or effect of interest, e.g., reducing one or more symptoms or manifestations of a disease or condition or modulating an immune response. In some embodiments a composition administered to a subject comprises up to about 10¹⁴ cells, e.g., about 10³, 10⁴, 10⁵, 10⁶, 10⁷, 10⁸, 10⁹, 10¹⁰, 10¹¹, 10¹², 10¹³ or 10¹⁴ cells, or any intervening number or range.

In another aspect, the composition of the present aspect may comprise a sortase and a sortase substrate but without red blood cells. The composition will be administered to the circulatory system in a subject and upon contacting red blood cells in vivo, the sortase conjugates the sortase substrate to at least one endogenous, non-engineered membrane protein of the red blood cells by a sortase-mediated reaction as described herein. In this form of composition, there will be no risk of incompatibility of red blood cells as well as other risks, such as bacterial or viruses contamination from donor cells. In some embodiments, the sortase has been further modified to enhance its stabilization in circulation by e.g., PEGylation or Fusion to Fc fragment and/or reduce its immunogenicity.

As used herein, the term “a physiologically acceptable carrier” is meant a solid or liquid filler, diluent or encapsulating substance that may be safely used in systemic administration. Depending upon the particular route of administration, a variety of carriers, diluent and excipients well known in the art may be used. These may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates, water and pyrogen-free water.

It will be appreciated by those skilled in the art that other variations of the embodiments described herein may also be practiced without departing from the scope of the invention. Other modifications are therefore possible.

Although the disclosure has been described and illustrated in exemplary forms with a certain degree of particularity, it is noted that the description and illustrations have been made by way of example only. Numerous changes in the details of construction and combination and arrangement of parts and steps may be made. Accordingly, such changes are intended to be included in the invention, the scope of which is defined by the claims.

### EXAMPLES

### Example 1

### Methods

Recombinant Protein Expression and Purification in E. coli

Mg SrtA (SEQ ID NO: 3), wt SrtA (SEQ ID NO: 1 with 25 amino acids removed from N-terminus) and eGFP-LPETG cDNA were cloned in pET vectors and transformed in E. coli BL21(DE3) cells for protein expression. Transformed cells were cultured at 37° C. until the OD₆₀₀ reaching 0.6-0.8 and then 500 μM IPTG were added for 4 hrs at 37° C. After that, cells were harvested by centrifugation and subjected to lysis by precooled lysis buffer (20 mM Tris-HCl, pH 7.8, 100 mM NaCl). The lysates were proceeded for sonication on ice (5 s on, 5 s off, 60 cycles, 25% power, Branson Sonifier 550 Ultrasonic Cell Disrupter). All supernatants were filtered by 0.22 μM filter after centrifugation at 14,000 g for 40 min at 4° C. Filtered supernatants were loaded onto HisTrap FF 1 mL column (GE Healthcare) connected to the AKTA design chromatography systems. The proteins were eluted with the elution buffer containing 20 mM Tris-HCl, pH 7.8, 100 mM NaCl and 300 mM imidazole. All eluted fractions were analyzed on a 12% SDS-PAGE gel.

### Wt SrtA or Mg SrtA-Mediated Enzymatic Labeling of Membrane Proteins

Reactions were performed in a total volume of 200 μL at 37° C. for 2 hrs in PBS buffer while being rotated at a speed of 10 rpm. The concentration of wt SrtA or mg SrtA was 20 μN1 and the biotin-LPETG (Synthesized by Beijing Scilight Biotechnology Led. Co.) or GFP-LPETG substrates were at the range of 500 μM. Human or mouse RBCs were washed twice with PBS before enzymatic reactions. The concentration of RBCs in the reaction was from 1×10⁹/mL. After the reaction, RBCs were washed three times and incubated with Streptavidin-phycoerythrin (PE) (BD Biosciences) at room temperature for 10 min before analyzed by Beckman Coulter CytoFLEX LX or Merck Amnis Image Stream MarkII.

### Enrichment of RBC Membrane Proteins

The biotin-labeled RBCs were resuspended in PBS and sonicated (10 s on, 10 s off, 3 cycles, 25% power, SONICS VCX150) on ice. Intact cells were removed by centrifugation at 4° C., 300×g for 15 min. Dried powder was obtained by freezing and lyophilizing then incubation with 50 mL of ice-cold 0.1 M sodium carbonate (pH=11) at 4° C. for 1 h with gentle rotation at a speed of 10 rpm. Membranous fractions were pelleted down by ultracentrifugation at 125,000×g at 4° C. for 1 h and then washed twice with Milli-Q water at the same speed for 30 mins. Then the samples were incubated with 2 mL of ice-cold 80% acetone for protein precipitation at −20° C. for 2 hrs. Membrane proteins were collected by centrifugation at 130,000×g at 4° C. for 15 mins. Membrane proteins samples were redissolved in 1% SDS and analyzed by gel electrophoresis using 12% SDS-PAGE.

### In-Gel Digestion

The whole gel was stained by coomassie blue (H₂0, 0.1% w/v Coomassie brilliant blue R250, 40% v/v methanol and 10% v/v acetic acid) at room temperature with gently shaking overnight then destained with the destaining solution (40% v/v methanol and 10% v/v acetic acid in water). The gel was rehydrated three times in distilled water at room temperature for 10 min with gentle agitation. The protein bands were cut out and further cut off into ca 1×1 mm² pieces, followed by reduction with 10 mM TCEP in 25 mM NH₄HCO₃ at 25° C. for 30 min, alkylation with 55 mM IAA in 25 mM NH₄HCO₃ solution at 25° C. in the dark for 30 min, and sequential digestion with rPNGase F at a concentration of 100 unit/ml at 37° C. for 4 hrs, and then digestion with trypsin at a concentration of 12.5 ng/mL at 37° C. overnight (1st digestion for 4 hrs and 2nd digestion for 12 hrs). Tryptic peptides were then extracted out from gel pieces by using 50% ACN/2.5% FA for three times and the peptide solution was dried under vacuum. Dry peptides were purified by Pierce C18 Spin Tips (Thermo Fisher, USA).

### Mass Spectrometry Analysis

Biognosys-11 iRT peptides (Biognosys, Schlieren, CH) were spiked into peptide samples at the final concentration of 10% prior to MS injection for RT calibration. Peptides were separated by Ultimate 3000 nanoLC-MS/MS system (Dionex LC-Packings, Thermo Fisher Scientific™, San Jose, USA) equipped with a 15 cm×75 μm ID fused silica column packed with 1.9 μm 120 Å C18. After injection, 500 ng peptides were trapped at 6 μL/min on a 20 mm×75 μm ID trap column packed with 3 μm 100 Å C18 aqua in 0.1% formic acid, 2% ACN. Peptides were separated along a 60 min 3-28% linear LC gradient (buffer A: 2% ACN, 0.1% formic acid (Fisher Scientific); buffer B: 98% ACN, 0.1% formic acid) at the flowrate of 300 nL/min (108 min inject-to-inject in total). Eluting peptides were ionized at a potential of +1.8 kV into a Q-Exactive HF mass spectrometer (Thermo Fisher Scientific™ San Jose, USA). Intact masses were measured at resolution 60,000 (at m/z 200) in the Orbitrap using an AGC target value of 3E6 charges and a maximum ion injection time of 80 ms. The top 20 peptide signals (charge-states higher than 2+ and lower than +6) were submitted to MS/MS in the HCD cell (1.6 amu isolation width, 27% normalized collision energy). MS/MS spectra were acquired at resolution 30,000 (at m/z 200) in the Orbitrap using an AGC target value of 1E5 charges, a maximum ion injection time of 100 ms. Dynamic exclusion was applied with a repeat count of 1 and an exclusion time of 30 s. The Maxquant (version 1.6.2.6) was used as a search engine with the fixed modification was cysteine (Cys) carbamidomethyl. and methionine (Met) oxidation as a variable modification. Variable modifications contained oxidation (M), deamidation (NQ), GX808-G-N, GX808-G-anywhere, GX808-K-sidechain. (for details, see Table 1). Other parameters were performed as default. Data was searched against the Swissprot Mouse database September 2018) and further filtered the data with FDR ≤1%.

### Results:

We first characterized the efficacy of mg SrtA-mediated labeling on RBC membranes. Wt SrtA was employed as the control for its recognition of three glycines at the N-terminus of proteins or peptides. Our results showed that >99% of natural mouse or human RBCs were biotin-labeled by mg SrtA in vitro. In contrast, no significant biotin signal was detected on the surface of mouse or human RBCs by wt SrtA nor the mock control group without enzyme (FIGS. 1A and 1B). Western-blot analysis also supported our flow cytometry results demonstrating mg SrtA-mediated biotin labeling of mouse RBCs (FIG. 1C). These results indicate that mg SrtA is more effective than wt SrtA in the engineering of natural red blood cells, and small molecules can be effectively labeled on red blood cells in this way. To further validate this finding, membrane proteins of natural mouse RBCs from the mg SrtA-labeled group or the mock control group were enriched by ultracentrifugation as described[6] (FIG. 1D). As expected, significant increases in biotin signals were detected in the mg SrtA-labeled group after the enrichment of RBC membrane proteins [6] (FIG. 1E). To assess the life-span of these surface modified RBCs in vivo, we next transfused biotin-LPETG tagged mouse RBCs, which were simultaneously labeled with a fluorescent dye DiR (1,1′-dioctadecyl-3,3,3′,3′-tetramethylindotricarbocyanine iodide), into wildtype recipient mice. The percentage of DiR and biotin positive RBCs in vivo was analyzed periodically. We found that biotin labeled RBCs by mg SrtA not only showed the same lifespan as the control groups but also remained 90% biotin positive during circulation (FIGS. 1F, 1G and 1H). Imaging analysis also showed convincing biotin signals on the cell surface and normal morphology of mg sortase-labeled RBCs (FIG. 1I). We also sortagged RBCs with eGFP-LPETG and transfused them into wildtype mice. As expected, RBCs conjugated with eGFP by mg SrtA but not by wt SrtA were detected in vivo, and the detected RBCs exhibited normal cellular morphology (FIGS. 1J and 1K). These results indicate that biological macromolecular substances such as proteins can be effectively labeled on red blood cells in this way. Taken together, our data suggests efficient labeling of peptides and proteins on the surface of natural RBCs mediated by mg SrtA both in vitro and in vivo.

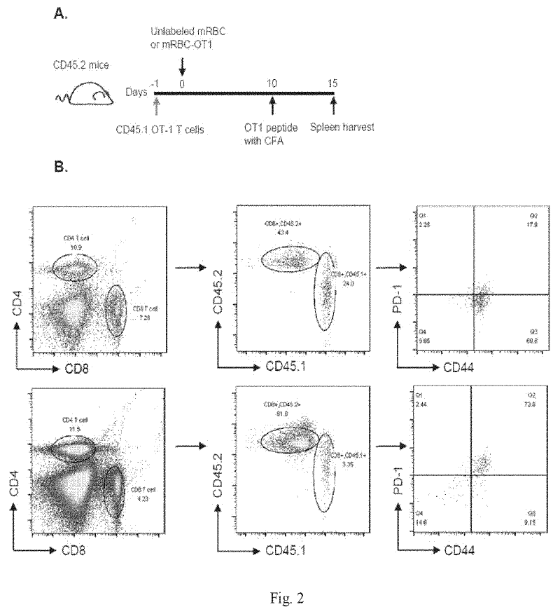

Previous studies have shown that specific-antigen bound RBCs are capable of inducing immunotolerance in several animal disease models[8]. In vitro generated mouse RBCs labeled with OT-1 peptide, which is an ovalbumin (OVA) epitope with SIINFEKL sequence, induce immunotolerances in CD8+ T cells with transgenic TCR recognizing H-2K^{b}-SIINFEKL in an autoimmune disease mouse model[8]. We adoptively transferred CD8⁺ CD45.1 T cells purified from OT1 TCR mice into CD45.2 recipient mice (FIG. 2A). After 24 hrs, same numbers of natural mouse RBCs modified with or without the OT-1 peptide by mg SrtA were injected into the recipient mice. The number of CD8⁺ CD45.1 T cells in the recipient mice receiving OT-1-RBC were ˜7 fold less compared to that in the mice injected with unmodified RBCs after the challenge with OT-1 peptides. Notably, the percentage of PD1⁺ CD8⁺ CD45.1⁺ T cells are over 4 times more in the mice receiving OT-1-RBC compared to that of recipient mice injected with natural RBCs. There is no change in the expression level of CD44 on the T cells in both groups which is consistent with previous studies[8][9]. These data suggested mg SrtA-modified RBCs carrying OT-1 peptide might induce OT-1 TCR T cell exhaustion but are more convenient and efficient for applications than previous strategies[8]. These results show that the use of this method to carry antigen proteins can effectively induce the production of immune tolerance, thereby providing new therapies for the treatment of clinical autoimmune diseases.

We next aim to identify the RBC membrane proteins serving as substrates for mg sortase mediated reaction. Biotin labeled RBCs by mg SrtA were analyzed by mass spectrometry (MS); a list of 122 candidate proteins potentially modified with biotin molecules on glycine (G) or the side chain of lysine (K) was detected (Table 1). 68 and 54 of these proteins were modified at glycine and the side chain of lysine, respectively (Tables 2 and 3). 18 of the identified proteins were detected with both modifications (Table 4). Among the total identified proteins, 22 proteins as shown in Table 5 were annotated as membrane proteins. For instance, the calcium-sensing receptor (CaSR), is a G-protein coupled receptor sensing calcium concentration in the circulation. Previous study has identified the presence of CaSR as a membrane protein on the RBC surface, which regulates the erythrocyte homeostasis[10]. Interestingly, biotin signals were detected at the G526 and K527 positions, neither of which is close to the N-terminus of CaSR. In addition, none of the rest 21 membrane proteins have biotin-modified glycine at the N-terminus, either. Therefore, we have identified membrane proteins including CaSR on RBC surface which might be covalently linked to biotin molecules.

Identification of biotin-labeled membrane proteins on RBCs was shown in Table 1. Biotin-labeled or natural RBC membrane proteins enriched from FIG. 1E were subjected to MS analysis. Enriched RBC membrane proteins were loaded into 1D gel electrophoresis for last in-gel digestion before being injected into MS instruments. The configuration on MaxQuant software were shown, which is the molecular weight (808 g/mol) increasing on the N-terminal and anywhere glycine and lysine, and the peptide searching was based on the UniProt protein database.

TABLE 1

| | | | | New | |
|---|---|---|---|---|---|
| Name | Composition | Position | Type | teminus | Specificities |
| GX808-G-N | C₃₆H₅₆O₁₁N₈S | Any N-term | Standard | None | G |
| GX808-G- | C₃₆H₅₆O₁₁N₈S | Anywhere | Standard | None | G |
| anywhere | | | | | |
| GX808-K- | C₃₆H₅₆O₁₁N₈S | Anywhere | Standard | None | K |
| side chain | | | | | |

A list of 68 protein candidates from RBCs modified with biotin-peptide on glycine(s) are shown in Table 2.

TABLE 2

| | | UniProt | Isoform | | | |
|---|---|---|---|---|---|---|
| No. | Protein names | ID | ID | Sequence | Length | Modifications |
| 1 | Extracellular calcium- | CASR | Q9QY96 | LFINEG | 1079 | G-anywhere |
| | sensing receptor | | | K | | |
| | (CaSR) (Parathyroid | | | | | |
| | cell calcium-sensing | | | | | |
| | receptor) (PCaR1) | | | | | |
| 2 | Ryanodine receptor 3 | RYR3 | A2AGL3 | NYMMS | 4863 | G-anywhere |
| | (RYR-3) (RyR3) | | | NGYK | | |
| | (Brain ryanodine | | | | | |
| | receptor-calcium | | | | | |
| | release channel) | | | | | |
| | (Brain-type ryanodine | | | | | |
| | receptor) (Type 3 | | | | | |
| | ryanodine receptor) | | | | | |
| 3 | Rap 1 GTPase- | RPGP1 | A2ALS5 | SSAIGIE | 663 | G-anywhere |
| | activating protein 1 | | | NIQEVQ | | |
| | (Rap1GAP) | | | EK | | |
| | (Rap1GAP1) (ARPP- | | | | | |
| | 90) | | | | | |
| 4 | Titin (EC 2.7.11.1) | TITIN | A2ASS6 | DGQVIS | 35213 | G-anywhere |
| | (Connectin) | | | TSTLPG | | |
| | | | | VQISFS | | |
| | | | | DGRAR | | |
| 5 | Inter alpha-trypsin | IT1H4 | A6X935 | GSRSQI | 942 | G-anywhere |
| | inhibitor, heavy chain | | | PR | | |
| | 4 (ITI heavy chain H4) | | | | | |
| | (ITI-HC4) (Inter- | | | | | |
| | alpha-inhibitor heavy | | | | | |
| | chain 4) | | | | | |
| 6 | Trafficking protein | TPC11 | B2RXC1 | VSLAGS | 1133 | G-anywhere |
| | particle complex | | | NVFQIG | | |
| | subunit 11 | | | VQDFVP | | |
| | | | | FVQCK | | |
| 7 | Desmoplakin (DP) | DESP | E9Q557 | NSQGSE | 2883 | G-anywhere |
| | | | | MFGDD | | |
| | | | | DKRR | | |
| 8 | Tumor protein D53 | TPD53 | O54818 | LGMNL | 204 | G-anywhere |
| | (mD53) (Tumor | | | MNELK | | |
| | protein D52-like 1) | | | | | |
| 9 | Inactive serine protease | PRS39 | O70169 | IYGGQI | 367 | G-anywhere |
| | 39 (Inactive testicular | | | AK | | |
| | serine protease 1) | | | | | |
| 10 | Lysine-specific | KDM6A | O70546 | QTLAN | 1401 | G-anywhere |
| | demethylase 6A (EC | | | GPFSAG | | |
| | 1.14.11.-) (Histone | | | HVPCST | | |
| | demethylase UTX) | | | SR | | |
| | (Ubiquitously | | | | | |
| | transcribed TPR | | | | | |
| | protein on the X | | | | | |
| | chromosome) | | | | | |
| | (Ubiquitously | | | | | |
| | transcribed X | | | | | |
| | chromosome | | | | | |
| | tetratricopeptide repeat | | | | | |
| | protein) | | | | | |
| 11 | Histone-lysine N- | SETB1 | O88974 | QGGQL | 1307 | G-anywhere |
| | methyltransferase | | | RTRPN | | |
| | SETDB1 (EC 2.1.1.-) | | | MGAVR | | |
| | (ERG-associated | | | | | |
| | protein with SET | | | | | |
| | domain) (ESET) (SET | | | | | |
| | domain bifurcated 1) | | | | | |
| 12 | NF-kappa-B inhibitor- | IKBL1 | O88995 | CPSAM | 381 | G-anywhere |
| | like protein 1 (Inhibitor | | | GIK | | |
| | of kappa B-like | | | | | |
| | protein) (I-kappa-B- | | | | | |
| | like protein) | | | | | |
| | (IkappaBL) (Nuclear | | | | | |
| | factor of kappa light | | | | | |
| | polypeptide gene | | | | | |
| | enhancer in B-cells | | | | | |
| | inhibitor-like 1) | | | | | |
| 13 | Vesicle transport | VT11A | O89116 | ILTGML | 217 | G-anywhere |
| | through interaction | | | RR | | |
| | with t-SNAREs | | | | | |
| | homolog 1A (Vesicle | | | | | |
| | transport v-SNARE | | | | | |
| | protein Vti1-like 2) | | | | | |
| | (Vti1-rp2) | | | | | |
| 14 | Fructose-bisphosphate | ALDOA | P05064 | LQSIGT | 364 | G-anywhere |
| | aldolase A (EC | | | ENTEEN | | |
| | 4.1.2.13) (Aldolase 1) | | | R | | |
| | (Muscle-type aldolase) | | | | | |
| 15 | T-cell surface | CD3G | P11942 | NTWNL | 182 | G-anywhere |
| | glycoprotein CD3 | | | GNNAK | | |
| | gamma chain (T-cell | | | | | |
| | receptor T3 gamma | | | | | |
| | chain) (CD antigen | | | | | |
| | CD3g) | | | | | |
| 16 | Medium-chain specific | ACAD | P45952 | ELNMG | 421 | G-anywhere |
| | acyl-CoA | M | | QR | | |
| | dehydrogenase, | | | | | |
| | mitochondrial | | | | | |
| | (MCAD) (EC 1.3.8.7) | | | | | |
| 17 | Sulfotransferase 1E1 | ST1E1 | P49891 | EGDVE | 295 | G-anywhere |
| | (ST1E1) (EC 2.8 2.4) | | | KCKED | | |
| | (Estrogen | | | AIFNR | | |
| | sulfotransferase, testis | | | | | |
| | isoform) | | | | | |
| | (Sulfotransferase, | | | | | |
| | estrogen-preferring) | | | | | |
| 18 | P2X purinoceptor 1 | P2RX1 | P51576 | NLSPGF | 399 | G-anywhere |
| | (P2X1) (ATP receptor) | | | NFR | | |
| | (Purinergic receptor) | | | | | |
| 19 | Scavenger receptor | C163A | Q2VLH6 | FQGKW | 1121 | G-anywhere |
| | cysteine-rich type 1 | | | GTVCD | | |
| | protein Ml 30 (CD | | | DNFSK | | |
| | antigen CD 163) | | | | | |
| | +Cleaved into: Soluble | | | | | |
| | CD 163 (sCD 163)1 | | | | | |
| 20 | RUN and FYVE | RUFY4 | Q3TYX8 | VEGKGS | 563 | G-anywhere |
| | domain-containing | | | LSGTED | | |
| | protein 4 | | | QRTTEG | | |
| | | | | IQK | | |
| 21 | Coiled-coil domain- | CC177 | Q3UHB | QEGQL | 706 | G-anywhere |
| | containing protein 177 | | 8 | QREK | | |
| 22 | Lysine-specific | KDM5A | Q3UXZ9 | TDIGVY | 1690 | G-anywhere |
| | demethylase 5A (EC | | | GSGKN | | |
| | 1.14.11.-) (Histone | | | R | | |
| | demethylase | | | | | |
| | JARID1A) (Jumonji/ | | | | | |
| | ARID domain- | | | | | |
| | containing protein 1A) | | | | | |
| | (Retinoblastoma- | | | | | |
| | binding protein 2) | | | | | |
| | (RBBP-2) | | | | | |
| 23 | Down syndrome cell | DSCL1 | Q4VA61 | DGQVII | 2053 | G-anywhere |
| | adhesion molecule-like | | | SGSGVT | | |
| | protein 1 homolog | | | IESK | | |
| 24 | C2 domain-containing | C2CD3 | Q52KB6 | GLPQDL | 2323 | G-anywhere |
| | protein 3 (Protein | | | DLMQK | | |
| | hearty) | | | | | |
| 25 | Vacuolar protein | VP13A | Q5H8C4 | GVAAM | 3166 | G-anywhere |
| | sorting-associated | | | TMDED | | |
| | protein 13A (Chorea- | | | YQQK | | |
| | acanthocytosis protein | | | | | |
| | homolog) (Chorein) | | | | | |
| 26 | Protein KIBRA | KIBRA | Q5SXA9 | TQKAE | 1104 | G-anywhere |
| | (Kidney and brain | | | GGSRLQ | | |
| | protein) (KIBRA) | | | ALR | | |
| | (WW domain- | | | | | |
| | containing protein 1) | | | | | |
| 27 | DNA polymerase zeta | REV3L | Q61493 | GNASH | 3122 | G-anywhere |
| | catalytic subunit (EC | | | ATGLFK | | |
| | 2.7.7.7) (Protein | | | | | |
| | reversionless 3-like) | | | | | |
| | (REV3-like) (Seizure- | | | | | |
| | related protein 4) | | | | | |
| 28 | Interferon-i nduced | IFIT3 | Q64345 | MGEEA | 403 | G-anywhere |
| | protein with | | | EGER | | |
| | tetratri copeptide | | | | | |
| | repeats 3 (IFIT-3) | | | | | |
| | (Glucocorticoid- | | | | | |
| | attenuated response | | | | | |
| | gene 49 protein) | | | | | |
| | (GARG-49) (P49) | | | | | |
| | (IRG2) | | | | | |
| 29 | Potassium-transporting | ATP4A | Q64436 | ILSAQG | 1033 | G-anywhere |
| | ATPase alpha chain 1 | | | CK | | |
| | (EC 7.2.2.19) (Gastric | | | | | |
| | H(+)/K(+) ATPase | | | | | |
| | subunit alpha) (Proton | | | | | |
| | pump) | | | | | |
| 30 | E3 ubiquitin-protein | SH3R1 | Q69ZI1 | LLSGAS | 892 | G-anywhere |
| | ligase SH3RF1 (EC | | | TKR | | |
| | 2.3.2.27) (Plenty of | | | | | |
| | SH3s) (Protein POSH) | | | | | |
| | (RING-type E3 | | | | | |
| | ubiquitin transferase | | | | | |
| | SH3RF1) (SH3 | | | | | |
| | domain-containing | | | | | |
| | RING finger protein 1) | | | | | |
| | (SH3 multiple domains | | | | | |
| | protein 2) | | | | | |
| 31 | Tubulin epsilon and | TEDC2 | Q6GQV0 | VLGTRS | 436 | G-anywhere |
| | delta complex protein | | | TK | | |
| | 2 | | | | | |
| 32 | FERM domain- | FRMD5 | Q6P5H6 | GPQLQQ | 517 | G-anywhere |
| | containing protein 5 | | | QQWK | | |
| 33 | Vacuolar ATPase | VMA21 | Q78T54 | QWREG | 101 | G-anywhere |
| | assembly integral | | | KQD | | |
| | membrane protein | | | | | |
| | Vma21 | | | | | |
| 34 | APC membrane | AMER1 | Q7TS75 | LFGGKK | 1132 | G-anywhere |
| | recruitment protein 1 | | | | | |
| | (Amerl) (Protein | | | | | |
| | FAM123B) | | | | | |
| 35 | Serine/threonine- | MRCK | Q7TT50 | DIKPDN | 1713 | G-anywhere |
| | protein kinase MRCK | B | | VLLDV | | |
| | beta (EC 2.7.11.1) | | | NGHIR | | |
| | (CDC42-binding | | | | | |
| | protein kinase beta) | | | | | |
| | (DMPK-like beta) | | | | | |
| | (Myotonic dystrophy | | | | | |
| | kinase-related CDC42- | | | | | |
| | binding kinase beta) | | | | | |
| | (MRCK beta) | | | | | |
| | (Myotonic dystrophy | | | | | |
| | protein kinase-like | | | | | |
| | beta) | | | | | |
| 36 | Uncharacterized | CJ062 | Q80Y39 | EMQRES | 304 | G-anywhere |
| | protein C10orf62 | | | GK | | |
| | homolog | | | | | |
| 37 | Dual specificity | DYRK4 | Q8BI55 | NINNNR | 632 | G-anywhere |
| | tyrosine- | | | GGKR | | |
| | phosphorylation- | | | | | |
| | regulated kinase 4 (EC | | | | | |
| | 2.7.12.1) | | | | | |
| 38 | Engulfment and cell | ELMOl | Q8BPU7 | GALKQ | 727 | G-anywhere |
| | motility protein 1 | | | NK | | |
| | (Protein ced-12 | | | | | |
| | homolog) | | | | | |
| 39 | Anaphase-promoti ng | APC5 | Q8BTZ4 | GRAMF | 740 | G-anywhere |
| | complex subunit 5 | | | LVSK | | |
| | (APC5) (Cyclosome | | | | | |
| | subunit 5) | | | | | |
| 40 | RNA-binding protein | RBM34 | Q8C5L7 | LNNSEL | 442 | G-anywhere |
| | 34 (RNA-binding | | | MGR | | |
| | motif protein 34) | | | | | |
| 41 | E3 ubiquitin-protein | ITCH | Q8C863 | TLNKPV | 864 | G-anywhere |
| | ligase Itchy (EC | | | GLK | | |
| | 2.3.2.26) (HECT-type | | | | | |
| | E3 ubiquitin | | | | | |
| | transferase Itchy | | | | | |
| | homolog) | | | | | |
| 42 | Coiled-coil domain- | CC159 | Q8C963 | WSTEQE | 411 | G-anywhere |
| | containing protein 159 | | | LYGAL | | |
| | | | | AQGLQ | | |
| | | | | GLQK | | |
| 43 | Death-inducer | DIDO1 | Q8C9B9 | SPAFEG | 2256 | G-anywhere |
| | obliterator 1 (DIO-1) | | | RQR | | |
| | (Death-associated | | | | | |
| | transcription factor 1) | | | | | |
| | (DATF-1) | | | | | |
| 44 | Coiled-coil domain- | CD158 | Q8CDI6 | ILRELD | 1109 | G-anywhere |
| | containing protein 158 | | | TEISFLK | | |
| | | | | GR | | |
| 45 | Structural maintenance | SMC4 | Q8CG47 | IFNLSG | 1286 | G-anywhere |
| | of chromosomes | | | GEK | | |
| | protein 4 (SMC protein | | | | | |
| | 4) (SMC-4) | | | | | |
| | (Chromosome- | | | | | |
| | associated polypeptide | | | | | |
| | C) (XCAP-C homolog) | | | | | |
| 46 | CD209 antigen-like | C209B | Q8CJ91 | IPISQGK | 325 | G-anywhere |
| | protein B (DC-SIGN- | | | | | |
| | related protein 1) (DC- | | | | | |
| | SIGNR1) (OtB7) (CD | | | | | |
| | antigen CD209) | | | | | |
| 47 | F-box DNA helicase 1 | FBH1 | Q8K2I9 | GINISNR | 1042 | G-anywhere |
| | (EC 3.6.4.12) (F-box | | | | | |
| | only protein 18) | | | | | |
| 48 | Serine dehydratase-like | SDSL | Q8R238 | IQLGCS | 329 | G-anywhere |
| | (EC 4.3.1.17) (L-serine | | | | | |
| | deaminase) (L-serine | | | | | |
| | dehydratase/L- | | | | | |
| | threonine deaminase) | | | | | |
| | (L-threonine | | | | | |
| | dehydratase) (TDH) | | | | | |
| | (EC 4.3.1.19) (SDH) | | | | | |
| 49 | Ribosome-releasing | RRF2M | Q8R2Q4 | 1LYYSG | 779 | G-anywhere |
| | factor 2, mitochondrial | | | YTR | | |
| | (RRF2mt) (Elongation | | | | | |
| | factor G 2, | | | | | |
| | mitochondrial) (EF- | | | | | |
| | G2mt) (mEF-G 2) | | | | | |
| 50 | OTU domain- | OTU7A | Q8R554 | AAMQG | 926 | G-anywhere |
| | containing protein 7A | | | ER | | |
| | (EC 3.4.19.12) (Zinc | | | | | |
| | finger protein Cezanne | | | | | |
| | 2) | | | | | |
| 51 | Leucine-rich repeat- | LRC14 | Q8VC16 | ELSMGS | 493 | G-anywhere |
| | containing protein 14 | | | SLLSGR | | |
| 52 | Neurotrophin receptor- | NR1F2 | Q921B4 | NQQLGS | 824 | G-anywhere |
| | interacting factor 2 | | | EQGKT | | |
| | (Zinc finger protein | | | QTSR | | |
| | 369) | | | | | |
| 53 | Electron transfer | ETFD | Q921G7 | GIATND | 616 | G-anywhere |
| | flavoprotein- | | | VGIQK | | |
| | ubiquinone | | | | | |
| | oxidoreductase, | | | | | |
| | mitochondrial (ETF- | | | | | |
| | QO) (ETF-ubiquinone | | | | | |
| | oxidoreductase) (EC | | | | | |
| | 1.5.5.1) (Electron- | | | | | |
| | transferring- | | | | | |
| | flavoprotein | | | | | |
| | dehydrogenase) (ETF | | | | | |
| | dehydrogenase) | | | | | |
| 54 | Polypeptide N- | GLT11 | Q921L8 | LMKCH | 608 | G-anywhere |
| | acetylgalactosaminyl | | | GSGGSQ | | |
| | transferase 11 (EC | | | QWTFG | | |
| | 2.4.1.41) (Polypeptide | | | K | | |
| | GalNAc transferase | | | | | |
| | 11) (GalNAc-T11) (pp- | | | | | |
| | GaNTase 11) (Protein- | | | | | |
| | UDP | | | | | |
| | acetylgalactosaminyl | | | | | |
| | transferase 11) (UDP- | | | | | |
| | GalNAc:polypeptide | | | | | |
| | N- | | | | | |
| | acetylgalactosaminyl | | | | | |
| | transferase 11) | | | | | |
| 55 | TOM 1-like protein 1 | TM1L1 | Q923U0 | LYKTGR | 474 | G-anywhere |
| | (Src-activating and | | | EMQER | | |
| | signaling molecule | | | | | |
| | protein) (Target of | | | | | |
| | Myb-like protein 1) | | | | | |
| 56 | Aconitate hydratase, | ACON | Q99KI0 | YLSKTG | 780 | G-anywhere |
| | mitochondrial | | | R | | |
| | (Aconitase) (EC | | | | | |
| | 4.2.1.3) (Citrate hydro- | | | | | |
| | lyase) | | | | | |
| 57 | Leucine-rich repeat- | LRC57 | Q9D1G5 | ELEGYD | 239 | G-anywhere |
| | containing protein 57 | | | K | | |
| 58 | Gamma- | GGCT | Q9D7X8 | LDFGNF | 188 | G-anywhere |
| | glutamylcyclo | | | QGKMS | | |
| | transferase (EC 4.3.2.9) | | | ER | | |
| 59 | Cyclin-L2 (Cyclin | CCNL2 | Q9JJA7 | ERTDNS | 518 | G-anywhere |
| | Ania-6b) (Paneth cell- | | | GKYK | | |
| | enhanced expression | | | | | |
| | protein) (PCEE) | | | | | |
| 60 | E3 SUMO-protein | PIAS4 | Q9JM05 | YLNGL | 507 | G-anywhere |
| | ligase P1AS4 (EC | | | GR | | |
| | 2.3.2.27) (PIASy) | | | | | |
| | (Protein inhibitor of | | | | | |
| | activated ST AT protein | | | | | |
| | 4) (Protein inhibitor of | | | | | |
| | activated ST AT protein | | | | | |
| | gamma) (P1AS- | | | | | |
| | gamma) (RING-type | | | | | |
| | E3 ubiquitin | | | | | |
| | transferase PIAS4) | | | | | |
| 61 | Calmodulin-4 | CALM4 | Q9JM83 | VADVD | 148 | G-anywhere |
| | (Calcium-binding | | | QDGK | | |
| | protein Dd112) | | | | | |
| 62 | PDZ domain- | PDZD4 | Q9QY39 | GCNMC | 772 | G-anywhere |
| | containing protein 4 | | | VVQK | | |
| | (PDZ domain- | | | | | |
| | containing RING | | | | | |
| | finger protein 4-like | | | | | |
| | protein) | | | | | |
| 63 | Short transient receptor | TRPC2 | Q9R244 | EGLTLP | 1172 | G-anywhere |
| | potential channel 2 | | | VPFNILP | | |
| | (TrpC2) (Transient | | | SPK | | |
| | receptor protein 2) | | | | | |
| | (TRP-2) (mTrp2) | | | | | |
| 64 | A-kinase anchor | AKA12 | Q9WTQ | ELEVPV | 1684 | G-anywhere |
| | protein 12 (AKAP-12) | | 5 | HTGPNS | | |
| | (Germ cell lineage | | | QKTAD | | |
| | protein gercelin) (Src- | | | LTR | | |
| | suppressed C kinase | | | | | |
| | substrate) (SSeCKS) | | | | | |
| 65 | ATP-dependent 6- | PFKAP | Q9WUA | GNQAV | 784 | G-anywhere |
| | phosphofructokinase, | | 3 | R | | |
| | platelet type (ATP- | | | | | |
| | PFK) (PFK-P) (EC | | | | | |
| | 2.7.1.11) (6- | | | | | |
| | phosphofructokinase | | | | | |
| | type C) | | | | | |
| | (Phosphofructo-1- | | | | | |
| | kinase isozyme C) | | | | | |
| | (PFK-C) | | | | | |
| | (Phosphohexokinase) | | | | | |
| 66 | Katanin p60 ATPase- | KTNA1 | Q9WV8 | GREEKN | 491 | G-anywhere |
| | containing subunit A1 | | 6 | K | | |
| | (Katanin p60 subunit | | | | | |
| | A1) (EC 5.6.1.1) | | | | | |
| | (Lipotransin) (p60 | | | | | |
| | katanin) | | | | | |
| 67 | R-spondin-1 (Cysteine- | RSPOl | Q9Z132 | KGGQG | 265 | G-anywhere |
| | rich and single | | | R | | |
| | thrombospondin | | | | | |
| | domain-containing | | | | | |
| | protein 3) (Cristin-3) | | | | | |
| | (mCristin-3) (Roof | | | | | |
| | plate-specific spondin- | | | | | |
| | 1) | | | | | |
| 68 | V-type proton ATPase | VATC1 | Q9Z1G3 | ASAYN | 382 | G-anywhere |
| | subunit C 1 (V-ATPase | | | NLKGN | | |
| | subunit C 1) (Vacuolar | | | LQNLER | | |
| | proton pump subunit C) | | | | | |

A list of 54 protein candidates from RBCs modified with biotin-peptide on the side chain of lysine(s) are shown in Table 3.

TABLE 3

| | | UniProt | Isoform | | | |
|---|---|---|---|---|---|---|
| No. | Protein names | ID | ID | Sequence | Length | Modifications |
| 1 | Extracellular calcium- | CASR | Q9QY96 | LFINEGK | 1079 | K-side chain |
| | sensing receptor | | | | | |
| | (CaSR) (Parathyroid | | | | | |
| | cell calcium-sensing | | | | | |
| | receptor) (PcaR1) | | | | | |
| 2 | Transcription factor | ZEP3 | A2A884 | GLPPMS | 2348 | K-side chain |
| | HIVEP3 (Human | | | VK | | |
| | immunodeficiency | | | | | |
| | virus type I enhancer- | | | | | |
| | binding protein 3 | | | | | |
| | homolog) (KB- | | | | | |
| | binding and | | | | | |
| | recognition | | | | | |
| | component) (Kappa-B | | | | | |
| | and V(D)J | | | | | |
| | recombination signal | | | | | |
| | sequences-binding | | | | | |
| | protein) (Kappa- | | | | | |
| | binding protein 1) | | | | | |
| | (KBP-1) | | | | | |
| | (Recombinant | | | | | |
| | component) | | | | | |
| | (Schnurri-3) (Zinc | | | | | |
| | finger protein ZAS3) | | | | | |
| 3 | Focadhesin | FOCAD | A2AKG | TYETNK | 1798 | K-side chain |
| | | | 8 | QPGLK | | |
| 4 | Arginine/serine-rich | RSRC2 | A2RTL5 | SQSAET | 376 | K-side chain |
| | coiled-coil protein 2 | | | WEK | | |
| 5 | E3 ubiquitin-protein | RN213 | E9Q555 | EIDVQY | 5152 | K-side chain |
| | ligase RNF213 (EC | | | K | | |
| | 2.3.2.27) (EC 3.6.4.-) | | | | | |
| | (Mysterin) (RING | | | | | |
| | finger protein 213) | | | | | |
| | (RJNG-type E3 | | | | | |
| | ubiquitin transferase | | | | | |
| | RNF213) | | | | | |
| 6 | Brefeldin A-inhibited | BIG1 | G3X9K3 | FLTSQQL | 1846 | K-side chain |
| | guanine nucleotide- | | | FK | | |
| | exchange protein 1 | | | | | |
| | (BIG 1) (Brefeldin A- | | | | | |
| | inhibited GEP 1) | | | | | |
| | (ADP-ribosylation | | | | | |
| | factor guanine | | | | | |
| | nucl eoti de-exchange | | | | | |
| | factor 1) | | | | | |
| 7 | Histone-lysine N- | NSD1 | 088491 | ETISAQ | 2588 | K-side chain |
| | methyltransferase, H3 | | | MVK | | |
| | lysine-36 and H4 | | | | | |
| | lysine-20 specific (EC | | | | | |
| | 2.1.1.-) (H3-K36- | | | | | |
| | HMTase) (H4-K20- | | | | | |
| | HMTase) (Nuclear | | | | | |
| | receptor-binding SET | | | | | |
| | domain-containing | | | | | |
| | protein 1) (NR- | | | | | |
| | binding SET domain- | | | | | |
| | containing protein) | | | | | |
| 7 | Histone-lysine N- | NSD1 | 088491 | LLNNMH | 2588 | K-side chain |
| | methyltransferase, H3 | | | EKTR | | |
| | lysine-36 and H4 | | | | | |
| | lysine-20 specific (EC | | | | | |
| | 2.1.1.-) (H3-K36- | | | | | |
| | HMTase) (H4-K20- | | | | | |
| | HMTase) (Nuclear | | | | | |
| | receptor-binding SET | | | | | |
| | domain-containing | | | | | |
| | protein 1) (NR- | | | | | |
| | binding SET domain- | | | | | |
| | containing protein) | | | | | |
| 8 | T-cell surface | CD3G | PI 1942 | NTWNLG | 182 | K-side chain |
| | glycoprotein CD3 | | | NNAK | | |
| | gamma chain (T-cell | | | | | |
| | receptor T3 gamma | | | | | |
| | chain) (CD antigen | | | | | |
| | CD3g) | | | | | |
| 9 | CD40 ligand (CD40- | CD40L | P27548 | KENSFE | 260 | K-side chain |
| | L) (T-cell antigen | | | MQR | | |
| | Gp39) (TNF-related | | | | | |
| | activation protein) | | | | | |
| | (TRAP) (Tumor | | | | | |
| | necrosis factor ligand | | | | | |
| | superfamily member | | | | | |
| | 5) (CD antigen | | | | | |
| | CD 154) +Cleaved | | | | | |
| | into: CD40 ligand, | | | | | |
| | membrane form; | | | | | |
| | CD40 ligand, soluble | | | | | |
| | form (sCD40L)l | | | | | |
| 10 | Sulfotransferase 1E1 | ST1E1 | P49891 | EGDVEK | 295 | K-side chain |
| | (ST1E1) (EC 2.8.2 4) | | | CKEDAIF | | |
| | (Estrogen | | | NR | | |
| | sulfotransferase, testis | | | | | |
| | isoform) | | | | | |
| | (Sulfotransferase, | | | | | |
| | estrogen-preferring) | | | | | |
| 11 | Solute carrier family | S12A2 | P55012 | RQAMKE | 1205 | K-side chain |
| | 12 member 2 | | | MS1DQA | | |
| | (Basolateral Na-K-Cl | | | R | | |
| | symporter) | | | | | |
| | (Bumetanide-sensitive | | | | | |
| | sodium-(potassium)- | | | | | |
| | chloride cotransporter | | | | | |
| | 2) | | | | | |
| 12 | 26S proteasome | PRS10 | P62334 | ALQDYR | 389 | K-side chain |
| | regulatory subunit | | | KK | | |
| | 10B (26S proteasome | | | | | |
| | AAA-ATPase subunit | | | | | |
| | RPT4) (Proteasome | | | | | |
| | 26S subunit ATPase | | | | | |
| | 6) (Proteasome | | | | | |
| | subunit p42) | | | | | |
| 13 | Adenylate cyclase | ADCY6 | Q01341 | LLLSVLP | 1165 | K-side chain |
| | type 6 (EC 4.6.1.1) | | | QHVAME | | |
| | (ATP pyrophosphate- | | | MK | | |
| | lyase 6) (Adenylate | | | | | |
| | cyclase type VI) | | | | | |
| | (Adenylyl cyclase 6) | | | | | |
| | (AC 6) (Ca(2+)- | | | | | |
| | inhibitable adenylyl | | | | | |
| | cyclase) | | | | | |
| 14 | Transcription factor | SOX13 | Q04891 | ILGSRW | 613 | K-side chain |
| | SOX-13 (SRY (Sex | | | KSMTNQ | | |
| | determining region | | | EK | | |
| | Y)-box 13) (mSoxl3) | | | | | |
| 15 | Leucine-rich repeat | LRIQ1 | Q0P5X1 | NQEKLM | 1673 | K-side chain |
| | and IQ domain- | | | AHKSEQ | | |
| | containing protein 1 | | | SR | | |
| 16 | von Willebrand factor | VWA3A | Q3UVV | EFQNDL | 1148 | K-side chain |
| | A domain-containing | | 9 | TGLIDEQ | | |
| | protein 3A | | | LSLKEK | | |
| 17 | Nesprin-3 (KASH | SYNE3 | Q4FZC9 | NQQLQR | 975 | K-side chain |
| | domain-containing | | | TEVDTG | | |
| | protein 3) (KASH3) | | | KK | | |
| | (Nuclear envelope | | | | | |
| | spectrin repeat protein | | | | | |
| | 3) | | | | | |
| 18 | Down syndrome cell | DSCL1 | Q4VA61 | DGQVIIS | 2053 | K-side chain |
| | adhesion molecule- | | | GSGVTIE | | |
| | like protein 1 | | | SK | | |
| | homolog | | | | | |
| 19 | Centrosome- | CP250 | Q60952 | QNEDYE | 2414 | K-side chain |
| | associated protein | | | KMVKAL | | |
| | CEP250 (250 k Da | | | R | | |
| | centrosomal protein) | | | | | |
| | (Cep250) | | | | | |
| | (Centrosomal Nek2- | | | | | |
| | associated protein 1) | | | | | |
| | (C-Napl) | | | | | |
| | (Centrosomal protein | | | | | |
| | 2) (Intranuclear | | | | | |
| | matrix protein) | | | | | |
| 20 | Cytochrome b-245 | CY24B | Q61093 | TIELQM | 570 | K-side chain |
| | heavy chain | | | KK | | |
| | (EC 1.-.-.-) (CGD91-phox) | | | | | |
| | (Cytochrome b(558) | | | | | |
| | subunit beta) | | | | | |
| | (Cytochrome b558 | | | | | |
| | subunit beta) (Heme- | | | | | |
| | binding membrane | | | | | |
| | glycoprotein | | | | | |
| | gp91phox) | | | | | |
| | (Neutrophil | | | | | |
| | cytochrome b 91 kDa | | | | | |
| | polypeptide) (gp91 -1) | | | | | |
| | (gp91-phox) (p22 | | | | | |
| | phagocyte B- | | | | | |
| | cytochrome) | | | | | |
| 21 | Heat shock protein | HS105 | Q61699 | NQQITH | 858 | K-side chain |
| | 105 kDa (42 degrees | | | ANNTVS | | |
| | C-HSP) (Heat shock | | | SFK | | |
| | 110 kDa protein) | | | | | |
| | (Heat shock-related | | | | | |
| | 100 kDa protein E7I) | | | | | |
| | (HSP-E7I) | | | | | |
| 22 | Tolloid-like protein 1 | TLL1 | Q62381 | LSEQSEK | 1013 | K-side chain |
| | (mTll) | | | NR | | |
| | (EC 3.4.24.-) | | | | | |
| 23 | E3 ubiquitin-protein | SH3R1 | Q69ZI1 | LLSGAST | 892 | K-side chain |
| | ligase SH3RF1 (EC | | | KR | | |
| | 2.3.2.27) (Plenty of | | | | | |
| | SH3s) (Protein | | | | | |
| | POSH) (RING-type | | | | | |
| | E3 ubiquitin | | | | | |
| | transferase SH3RF1) | | | | | |
| | (SH3 domain- | | | | | |
| | containing RING | | | | | |
| | finger protein 1) (SH3 | | | | | |
| | multiple domains | | | | | |
| | protein 2) | | | | | |
| 24 | Tubulin epsilon and | TEDC2 | Q6GQV | VLGTRS | 436 | K-side chain |
| | delta complex protein | | 0 | TK | | |
| | 2 | | | | | |
| 25 | Vacuolar ATPase | VMA21 | Q78T54 | QWREGK | 101 | K-side chain |
| | assembly integral | | | QD | | |
| | membrane protein | | | | | |
| | Vma21 | | | | | |
| 26 | Centrosomal protein | CE120 | Q7TSG1 | DQQNNK | 988 | K-side chain |
| | of 120 kDa (Cep 120) | | | PEIR | | |
| | (Coiled-coil domain- | | | | | |
| | containing protein | | | | | |
| | 100) | | | | | |
| 27 | Transcription | TAF1 | Q80UV9 | LKRNQE | 1891 | K-side chain |
| | initiation factor TF11D | | | K | | |
| | subunit 1 (EC | | | | | |
| | 2.3.1.48) (EC | | | | | |
| | 2.7.11.1) (Cell cycle | | | | | |
| | gene 1 protein) (TBP- | | | | | |
| | associated factor 250 | | | | | |
| | kDa) (p250) | | | | | |
| | (Transcription | | | | | |
| | initiation factor TF1LD | | | | | |
| | 250 kDa subunit) | | | | | |
| | (TAF(II)250) (TAFII- | | | | | |
| | 250) (TAFI1250) | | | | | |
| 28 | Carbohydrate | CHSTE | Q80V53 | LLSAYR | 376 | K-side chain |
| | sulfotransferase 14 | | | NK | | |
| | (EC 2.8.2.35) | | | | | |
| | (Dermatan 4- | | | | | |
| | sulfotransferase 1) | | | | | |
| | (D4ST-1) | | | | | |
| 29 | Multidrug resistance- | MRP9 | Q80WJ6 | LMNRFS | 1366 | K-side chain |
| | associated protein 9 | | | K | | |
| | (ATP-binding cassette | | | | | |
| | sub-family C member | | | | | |
| | 12) | | | | | |
| 30 | Uncharacterized | CJ062 | Q80Y39 | EMQRES | 304 | K-side chain |
| | protein C10orf62 | | | GK | | |
| | homolog | | | | | |
| 31 | Tenascin-N (TN-N) | TENN | Q80Z71 | LEEEMA | 1560 | K-side chain |
| | (Tenascin-W) (TN- | | | ELKEQC | | |
| | W) | | | NTNR | | |
| 32 | BRCA2-interacti ng | EMSY | Q8BMB0 | ITTIPMT | 1264 | K-side chain |
| | transcriptional | | | SK | | |
| | repressor EMSY | | | | | |
| 33 | Zinc finger protein | DZIP1 | Q8BMD2 | LNKKTS | 852 | K-side chain |
| | DZ1P1 (DAZ- | | | LR | | |
| | i nteracti ng protein 1 | | | | | |
| | homolog) | | | | | |
| 34 | Phosphatidylinositol | PK3CB | Q8BT19 | KMYEQE | 1064 | K-side chain |
| | 4,5-bisphosphate 3- | | | MIAIEAA | | |
| | kinase catalytic | | | INR | | |
| | subunit beta isoform | | | | | |
| | (PI3-kinase subunit | | | | | |
| | beta) (PI3K-beta) | | | | | |
| | (PI3Kbeta) (PtdIns-3- | | | | | |
| | kinase subunit beta) | | | | | |
| | (EC 2.7.1.153) | | | | | |
| | (Phosphatidylinositol | | | | | |
| | 4,5-bisphosphate 3- | | | | | |
| | kinase 110 kDa | | | | | |
| | catalytic subunit beta) | | | | | |
| | (PtdIns-3-kinase | | | | | |
| | subunit pi 10-beta) | | | | | |
| | (pi lObeta) | | | | | |
| 35 | Dynein heavy chain 3, | DYH3 | Q8BW9 | KMKFNL | 4083 | K-side chain |
| | axonemal (Axonemal | | 4 | K | | |
| | beta dynein heavy | | | | | |
| | chain 3) (Ciliary | | | | | |
| | dynein heavy chain 3) | | | | | |
| 36 | E3 ubiquitin-protein | ITCH | Q8C863 | ILNKPVG | 864 | K-side chain |
| | ligase Itchy (EC | | | LK | | |
| | 2.3.2.26) (HECT-type | | | | | |
| | E3 ubiquitin | | | | | |
| | transferase Itchy | | | | | |
| | homolog) | | | | | |
| 37 | MICOS complex | MIC60 | Q8CAQ | LEEKRTF | 757 | K-side chain |
| | subunit Mic60 | | 8 | DSAVAK | | |
| | (Mitochondrial inner | | | | | |
| | membrane protein) | | | | | |
| | (Mitofilin) | | | | | |
| 38 | Ras-related protein | RAB44 | Q8CB87 | VKNLLV | 973 | K-sidc chain |
| | Rab-44 | | | DNK | | |
| 39 | Leucine-rich repeat- | LRRC9 | Q8CDN | IEFLQQK | 1456 | K-side chain |
| | containing protein 9 | | 9 | | | |
| 40 | Structural | SMC4 | Q8CG47 | IFNLSGG | 1286 | K-side chain |
| | maintenance of | | | EK | | |
| | chromosomes protein | | | | | |
| | 4 (SMC protein 4) | | | | | |
| | (SMC-4) | | | | | |
| | (Chromosome- | | | | | |
| | associated | | | | | |
| | polypeptide C) | | | | | |
| | (XCAP-C homolog) | | | | | |
| 41 | CD209 antigen-like | C209B | Q8CJ91 | IPISQGK | 325 | K-side chain |
| | protein B (DC-S1GN- | | | | | |
| | related protein 1) | | | | | |
| | (DC-SIGNR1) (OtB7) | | | | | |
| | (CD antigen CD209) | | | | | |
| 42 | F-box DNA helicase | FBH1 | Q8K2I9 | YVTAAE | 1042 | K-side chain |
| | 1 (EC 3.6.4.12) (F- | | | DKELEA | | |
| | box only protein 18) | | | KIAVVE | | |
| | | | | K | | |
| 43 | Major intrinsically | MNAR1 | Q8K3V7 | CSVNNQ | 917 | K-side chain |
| | disordered Notch2- | | | QSK | | |
| | binding receptor 1 | | | | | |
| | (Membrane integral | | | | | |
| | NOTCH2-associated | | | | | |
| | receptor 1) (Protein | | | | | |
| | DD1) (Ubiquitination | | | | | |
| | and mTOR signaling | | | | | |
| | protein) | | | | | |
| 44 | Actin-related protein | ARP8 | Q8R2S9 | QNGLKM | 624 | K-side chain |
| | 8 | | | VDQA1W | | |
| | | | | SK | | |
| 45 | Leucine-rich repeat- | LRC14 | Q8VC16 | VAFMDK | 493 | K-side chain |
| | containing protein 14 | | | KTLVLR | | |
| 46 | Electron transfer | ETFD | Q921G7 | GIATND | 616 | K-side chain |
| | flavoprotein- | | | VGIQK | | |
| | ubiquinone | | | | | |
| | oxidoreductase, | | | | | |
| | mitochondrial (ETF- | | | | | |
| | QO) (ETF-ubiquinone | | | | | |
| | oxidoreductase) (EC | | | | | |
| | 1.5.5.1) (Electron- | | | | | |
| | transferring- | | | | | |
| | flavoprotein | | | | | |
| | dehydrogenase) (ETF | | | | | |
| | dehydrogenase) | | | | | |
| 47 | Tetratri copeptide | TTC14 | Q9CSP9 | NEAPEE | 761 | K-side chain |
| | repeat protein 14 | | | MLNSK | | |
| | (TPR repeat protein | | | | | |
| | 14) | | | | | |
| 48 | Ganima- | GGCT | Q9D7X8 | LDFGNF | 188 | K-side chain |
| | glutamylcyclotransfer | | | QGKMSE | | |
| | ase (EC 4.3.2 9) | | | R | | |
| 49 | LanC-like protein 2 | LANC2 | Q9JJK2 | SLSREER | 450 | K-side chain |
| | (Testis-specific | | | K | | |
| | adriamycin sensitivity | | | | | |
| | protein) | | | | | |
| 50 | Calmodulin-4 | CALM4 | Q9JM83 | VADVDQ | 148 | K-side chain |
| | (Calcium-binding | | | DGK | | |
| | protein Ddl 12) | | | | | |
| 51 | Plexin-Cl (Virus- | PLXC1 | Q9QZC2 | NQELCQ | 1574 | K-side chain |
| | encoded semaphorin | | | VAVEKS | | |
| | protein receptor) (CD | | | PK | | |
| | antigen CD232) | | | | | |
| 52 | Protein BEX1 (Brain- | BEX1 | Q9R224 | NLNMEN | 128 | K-side chain |
| | expressed X-linked | | | DHQKKE | | |
| | protein 1 homolog) | | | EK | | |
| | (Reduced expression | | | | | |
| | protein 3) (REX-3) | | | | | |
| 53 | Short transient | TRPC2 | Q9R244 | EGLTLP | 1172 | K-side chain |
| | receptor potential | | | VPFN1LP | | |
| | channel 2 (TrpC2) | | | SPK | | |
| | (Transient receptor | | | | | |
| | protein 2) (TRP-2) | | | | | |
| | (mTrp2) | | | | | |
| 54 | A-kinase anchor | AKA12 | Q9WTQ | ELEVPV | 1684 | K-side chain |
| | protein 12 (AKAP- | | 5 | HTGPNS | | |
| | 12) (Germ cell | | | QKTADL | | |
| | lineage protein | | | TR | | |
| | gercelin) (Src- | | | | | |
| | suppressed C kinase | | | | | |
| | substrate) (SSeCKS) | | | | | |

A list of 18 protein candidates from RBCs modified with biotin-peptide on glycine and the side chain of lysine were shown in Table 4.

TABLE 4

| | | UniProt | Isoform | | | |
|---|---|---|---|---|---|---|
| No. | Protein names | ID | ID | Sequence | Length | Modifications |
| 1 | Extracellular calcium- | CASR | Q9QY96 | LFINEGK | 1079 | G-anywhere |
| | sensing receptor | | | | | and K side |
| | (CaSR) (Parathyroid | | | | | chain |
| | cell calcium-sensing | | | | | |
| | receptor) (PCaR1) | | | | | |
| 2 | T-cell surface | CD3G | P11942 | NTWNLG | 182 | G-anywhere |
| | glycoproteinCD3 | | | NNAK | | and K side |
| | gamma chain (T-cell | | | | | chain |
| | receptor T3 gamma | | | | | |
| | chain) (CD antigen | | | | | |
| | CD3g) | | | | | |
| 3 | Sulfotransferase 1E1 | ST1E1 | P49891 | EGDVEK | 295 | G-anywhere |
| | (STIE1) (EC 2.8.2.4) | | | CKEDAIF | | and K side |
| | (Estrogen | | | NR | | chain |
| | sulfotransferase, testis | | | | | |
| | isoform) | | | | | |
| | (Sulfotransferase, | | | | | |
| | estrogen-preferring) | | | | | |
| 4 | Down syndrome cell | DSCL1 | Q4VA61 | DGQVIIS | 2053 | G-anywhere |
| | adhesion molecule- | | | GSGVTIE | | and K side |
| | like protein 1 | | | SK | | chain |
| | homolog | | | | | |
| 5 | E3 ubiquitin-protein | SH3R1 | Q69ZI1 | LLSGAST | 892 | G-anywhere |
| | ligase SH3RF1 (EC | | | KR | | and K side |
| | 2.3.2.27) (Plenty of | | | | | chain |
| | SH3s)(Protein | | | | | |
| | POSH) (RING-type | | | | | |
| | E3ubiquitin | | | | | |
| | transferase SH3RF1) | | | | | |
| | (SH3 domain- | | | | | |
| | containing RING | | | | | |
| | finger protein 1) (SH3 | | | | | |
| | multiple domains | | | | | |
| | protein 2) | | | | | |
| 6 | Tubulin epsilon and | TEDC2 | Q6GQV | VLGTRS | 436 | G-anywhere |
| | delta complex protein | | 0 | TK | | and K side |
| | 2 | | | | | chain |
| 7 | Vacuolar ATPase | VMA21 | Q78T54 | QWREGK | 101 | G-anywhere |
| | assembly integral | | | QD | | and K side |
| | membrane protein | | | | | chain |
| | Vma21 | | | | | |
| 8 | Uncharacterized | CJ062 | Q80Y39 | EMQRES | 304 | G-anywhere |
| | protein C10orf62 | | | GK | | and K side |
| | homolog | | | | | chain |
| 9 | E3 ubiquitin-protein | ITCH | Q8C863 | ILNKPVG | 864 | G-anywhere |
| | ligase Itchy (EC | | | LK | | and K side |
| | 2.3.2.26) (HECT-type | | | | | chain |
| | E3 ubiquitin | | | | | |
| | transferase Itchy | | | | | |
| | homolog) | | | | | |
| 10 | Structural | SMC4 | Q8CG47 | IFNLSGG | 1286 | G-anywhere |
| | maintenance of | | | EK | | and K side |
| | chromosomes protein | | | | | chain |
| | 4 (SMC protein 4) | | | | | |
| | (SMC-4) | | | | | |
| | (Chromosome- | | | | | |
| | associated | | | | | |
| | polypeptide C) | | | | | |
| | (XCAP-C homolog) | | | | | |
| 11 | CD209 antigen-like | C209B | Q8CJ91 | IPISQGK | 325 | G-anywhere |
| | protein B (DC-SIGN- | | | | | and K side |
| | related protein 1) | | | | | chain |
| | (DC-SIGNR1) (OtB7) | | | | | |
| | (CD antigen CD209) | | | | | |
| 12 | F-box DNA helicase | FBH1 | Q8K2I9 | GINISNR; | 1042 | G-anywhere |
| | 1 (EC 3.6.4.12) (F- | | | and | | and K side |
| | box only protein 18) | | | YVTAAE | | chain |
| | | | | DKELEA | | |
| | | | | KIA WE | | |
| | | | | K | | |
| 13 | Leucine-rich repeat- | LRC14 | Q8VC16 | ELSMGS | 493 | G-anywhere |
| | containing protein 14 | | | SLLSGR; | | and K side |
| | | | | and | | chain |
| | | | | VAFMDK | | |
| | | | | KTLVLR | | |
| 14 | Electron transfer | ETFD | Q921G7 | GIATND | 616 | G-anywhere |
| | flavoprotein- | | | VGIQK | | and K side |
| | ubiquinone | | | | | chain |
| | oxidoreductase, | | | | | |
| | mitochondrial (ETF- | | | | | |
| | QO) (ETF-ubiquinone | | | | | |
| | oxidoreductase) (EC | | | | | |
| | 1.5.5.1) (Electron- | | | | | |
| | transferring- | | | | | |
| | flavoprotein | | | | | |
| | dehydrogenase) (ETF | | | | | |
| | dehydrogenase) | | | | | |
| 15 | Gamma- | GGCT | Q9D7X8 | LDFGNF | 188 | G-anywhere |
| | glutamylcyclo | | | QGKMSE | | and K side |
| | transferase | | | R | | chain |
| | (EC 4.3.2.9) | | | | | |
| 16 | Calmodulin-4 | CALM4 | Q9JM83 | VADVDQ | 148 | G-anywhere |
| | (Calcium-binding | | | DGK | | and K side |
| | protein Dd112) | | | | | chain |
| 17 | Short transient | TRPC2 | Q9R244 | EGLTLP | 1172 | G-anywhere |
| | receptor potential | | | VPFNILP | | and K side |
| | channel 2 (TrpC2) | | | SPK | | chain |
| | (Transient receptor | | | | | |
| | protein 2) (TRP-2) | | | | | |
| | (mTrp2) | | | | | |
| 18 | A-kinase anchor | AKA12 | Q9WTQ | ELEVPV | 1684 | G-anywhere |
| | protein 12 (AKAP- | | 5 | HTGPNS | | and K side |
| | 12) (Germ cell | | | QKTADL | | chain |
| | lineage protein | | | TR | | |
| | gercelin)(Src- | | | | | |
| | suppressed C kinase | | | | | |
| | substrate) (SSeCKS) | | | | | |

A list of 22 membrane protein candidates from RBCs modified with biotin-peptide on glycine and the side chain of lysine were shown in Table 5.

TABLE 5

| | | | | | | Modification |
|---|---|---|---|---|---|---|
| | | UniProt | Isoform | | | typeand |
| No. | Protein names | ID | ID | Sequence | Length | position |
| 1 | Extracellular calcium- | CASR | Q9QY96 | LFINEGK | 1079 | G-anywhere |
| | sensing receptor | | | | | and K side |
| | (CaSR) (Parathyroid | | | | | chain; G526/ |
| | cell calcium-sensing | | | | | K527 |
| | receptor) (PCaRl) | | | | | |
| 2 | T-cell surface | CD3G | P11942 | NTWNLG | 182 | G-anywhere |
| | glycoprotein CD3 | | | NNAK | | and K side |
| | gamma chain (T-cell | | | | | chain; G158/ |
| | receptor T3 gamma | | | | | K162 |
| | chain) (CD antigen | | | | | |
| | CD3g) | | | | | |
| 3 | Down syndrome cell | DSCL1 | Q4VA61 | DGQVIIS | 2053 | G-anywhere |
| | adhesion molecule- | | | GSGVTIE | | and K side |
| | like protein 1 | | | SK | | chain; G698/ |
| | homolog | | | | | K706 |
| 4 | Short transient | TRPC2 | Q9R244 | EGLTLP | 1172 | G-anywhere |
| | receptor potential | | | VPFNILP | | and K side |
| | channel 2 (TrpC2) | | | SPK | | chain; G950/ |
| | (Transient receptor | | | | | K964 |
| | protein 2) (TRP-2) | | | | | |
| | (mTrp2) | | | | | |
| 5 | CD209 antigen-like | C209B | Q8CJ91 | IPISQGK | 325 | G-anywhere |
| | protein B (DC-SIGN- | | | | | and K side |
| | related protein 1) | | | | | chain; G110/ |
| | (DC-SIGNR1) (OtB7) | | | | | Kill |
| | (CD antigen CD209) | | | | | |
| 6 | E3 ubiquitin-protein | ITCH | Q8C863 | ILNKPVG | 864 | G-anywhere |
| | ligase Itchy (EC | | | LK | | and K side |
| | 2.3.2.26) (HECT-type | | | | | chain; K631/ |
| | E3 ubiquitin | | | | | G634 |
| | transferase Itchy | | | | | |
| | homolog) | | | | | |
| 7 | A-kinase anchor | AKA12 | Q9WTQ | ELEVPV | 1684 | G-anywhere |
| | protein 12 (AKAP-12) | | 5 | HTGPNS | | and K side |
| | (Germ cell lineage | | | QKTADL | | chain; G1259/ |
| | protein gercelin) (Src- | | | TR | | K1264 |
| | suppressed C kinase | | | | | |
| | substrate) (SSeCKS) | | | | | |
| 8 | Inter alpha-trypsin | ITIH4 | A6X935 | GSRSQIP | 942 | G-anywhere; |
| | inhibitor, heavy chain | | | R | | G642 |
| | 4 (ITI heavy chain | | | | | |
| | H4) (ITI-HC4) (Inter- | | | | | |
| | alpha-inhibitor heavy | | | | | |
| | chain 4) | | | | | |
| 9 | Potassium- | ATP4A | Q64436 | ILSAQGC | 1033 | G-anywhere; |
| | transporting ATPase | | | K | | G219 |
| | alpha chain 1 (EC | | | | | |
| | 7.2.2.19) (Gastric | | | | | |
| | H(+)/K(+) ATPase | | | | | |
| | subunit alpha) (Proton | | | | | |
| | pump) | | | | | |
| 10 | P2X purinoceptor 1 | P2RX1 | P51576 | NLSPGF | 399 | G-anywhere; |
| | (P2X1) (ATP | | | NFR | | G288 |
| | receptor) (Purinergic | | | | | |
| | receptor) | | | | | |
| 11 | Ryanodine receptor 3 | RYR3 | A2AGL3 | NYMMS | 4863 | G-anywhere; |
| | (RYR-3) (RyR3) | | | NGYK | | G962 |
| | (Brain ryanodine | | | | | |
| | receptor-calcium | | | | | |
| | release channel) | | | | | |
| | (Brain-type ryanodine | | | | | |
| | receptor) (Type 3 | | | | | |
| | ryanodine receptor) | | | | | |
| 12 | Scavenger receptor | C163A | Q2VLH6 | FQGKWG | 1121 | G-anywhere; |
| | cysteine-rich type 1 | | | TVCDDN | | G180 |
| | protein Ml30 (CD | | | FSK | | |
| | antigen CD 163) | | | | | |
| | [Cleaved into: Soluble | | | | | |
| | CD 163 (sCD163)] | | | | | |
| 13 | APC membrane | AMER1 | Q7TS75 | LFGGKK | 1132 | G-anywhere; |
| | recruitment protein 1 | | | | | G61 |
| | (Amerl) (Protein | | | | | |
| | FAM123B) | | | | | |
| 14 | Serine/threonine- | MRCKB | Q7TT50 | DIKPDN | 1713 | G-anywhere; |
| | protein kinase MRCK | | | VLLDVN | | G212 |
| | beta (EC 2.7.11.1) | | | GHIR | | |
| | (CDC42-binding | | | | | |
| | protein kinase beta) | | | | | |
| | (DMPK-like beta) | | | | | |
| | (Myotonic dystrophy | | | | | |
| | kinase-related | | | | | |
| | CDC42-binding | | | | | |
| | kinase beta) (MRCK | | | | | |
| | beta) (Myotonic | | | | | |
| | dystrophy protein | | | | | |
| | kinase-like beta) | | | | | |
| 15 | Engulfment and cell | ELMO1 | Q8BPU7 | GALKQN | 727 | G-anywhere; |
| | motility protein 1 | | | K | | G629 |
| | (Protein ced-12 | | | | | |
| | homolog) | | | | | |
| 16 | Desmoplakin (DP) | DESP | E9Q557 | NSQGSE | 2883 | G-anywhere; |
| | | | | MFGDDD | | G608 |
| | | | | KRR | | |
| 17 | CD40 ligand (CD40- | CD40L | P27548 | KENSFE | 260 | K-side chain; |
| | L) (T-cell antigen | | | MQR | | K106 |
| | Gp39) (TNF-related | | | | | |
| | activation protein) | | | | | |
| | (TRAP) (Tumor | | | | | |
| | necrosis factor ligand | | | | | |
| | superfamily member | | | | | |
| | 5) (CD antigen | | | | | |
| | CD 154) [Cleaved | | | | | |
| | into: CD40 ligand, | | | | | |
| | membrane form; | | | | | |
| | CD40 ligand, soluble | | | | | |
| | form (sCD40L)] | | | | | |
| 18 | Solute carrier family | S12A2 | P55012 | RQAMKE | 1205 | K-side chain; |
| | 12 member 2 | | | MSIDQA | | K826 |
| | (Basolateral Na-K-Cl | | | R | | |
| | symporter) | | | | | |
| | (Bumetanide-sensitive | | | | | |
| | sodium-(potassium)- | | | | | |
| | chloride cotransporter | | | | | |
| | 2) | | | | | |
| 19 | Adenylate cyclase | ADCY6 | Q01341 | LLLSVLP | 1165 | K-side chain; |
| | type 6 (EC 4.6.1.1) | | | QHVAME | | K353 |
| | (ATP pyrophosphate- | | | MK | | |
| | lyase 6) (Adenylate | | | | | |
| | cyclase type VI) | | | | | |
| | (Adenylyl cyclase 6) | | | | | |
| | (AC6) (Ca(2+)- | | | | | |
| | inhibitable adenylyl | | | | | |
| | cyclase) | | | | | |
| 20 | Cytochrome b-245 | CY24B | Q61093 | TIELQM | 570 | K-side chain; |
| | heavy chain (EC 1.-.- | | | KK | | K313/K314 |
| | .-) (CGD91-phox) | | | | | |
| | (Cytochrome b(558) | | | | | |
| | subunit beta) | | | | | |
| | (Cytochrome b558 | | | | | |
| | subunit beta) (Heme- | | | | | |
| | binding membrane | | | | | |
| | glycoprotein | | | | | |
| | gp91phox) | | | | | |
| | (Neutrophil | | | | | |
| | cytochrome b 91 kDa | | | | | |
| | polypeptide) (gp91-l) | | | | | |
| | (gp91-phox)(p22 | | | | | |
| | phagocyte B- | | | | | |
| | cytochrome) | | | | | |
| 21 | Major intrinsically | MNAR1 | Q8K3V7 | CSVNNQ | 917 | K-side chain; |
| | disordered Notch2- | | | QSK | | K79 |
| | binding receptor 1 | | | | | |
| | (Membrane integral | | | | | |
| | NOTCH2-associated | | | | | |
| | receptor 1) (Protein | | | | | |
| | DD1) (Ubiquitination | | | | | |
| | and mTOR signaling | | | | | |
| | protein) | | | | | |
| 22 | Plexin-Cl (Virus- | PLXC1 | Q9QZC2 | NQELCQ | 1574 | K-side chain; |
| | encoded semaphorin | | | VAVEKS | | K642 |
| | protein receptor) (CD | | | PK | | |
| | antigen CD232) | | | | | |

### Example 2

Mg SrtA-Mediated Enzymatic Labeling of RBC Membrane Proteins with ACE2-Fc (Fc Fragment)

Reactions were performed in a total volume of 200 μL at 37° C. for 2 hrs in PBS buffer while being rotated at a speed of 10 rpm. The concentration of truncated mg SrtA (SEQ ID NO: 27) was 10 μM and the concentration of ACE2-Fc-LPETG substrates was 50 μM. Mouse RBCs were washed twice with PBS before the enzymatic reaction. The concentration of RBCs in the reaction was 1×10⁹/mL. After the reaction, RBCs were washed three times and incubated with Anti-ACE2 AF700 at room temperature for 10 min before analyzed by Beckman Coulter CytoFLEX LX.

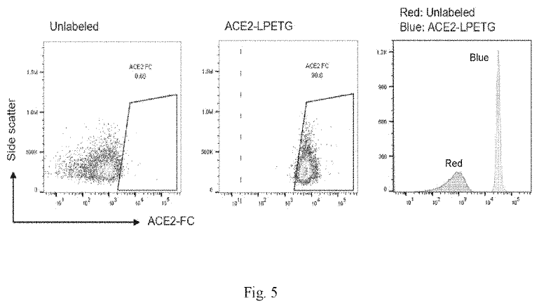

As shown in FIG. 5, the labeling efficiency of ACE2-Fc-LPETG on the surface of natural RBCs was detected by flow cytometry. RED: Unlabled RBCs; BLUE: RBCs labeled with ACE2-Fc-LPETG. Histograms showed ACE2-Fc-LEPTG signals on the RBCs' surface after their incubation with ACE2-Fc-LPETG.

To assess the life-span of these surface modified RBCs in vivo, we next transfused ACE2-FC-LEPTG tagged mouse RBCs (Dosage: 1×10⁹/mouse), which were simultaneously labeled by a fluorescent dye cell trace CF SE, into recipient mice. The percentage of CFSE and ACE2-Fc-LEPTG positive RBCs in vivo was analyzed periodically. Specifically, the percentage of ACE2-FC positive cells in the circulation and the label stability of these RBCs in different days. (a) Recipient mice were bled at indicated days post transfusion. CFSE positive cells indicate the percentage of transfused RBCs in the circulation. (b) CFSE positive RBCs from the blood samples of the above experiments were analyzed for measuring the label stability of these ACE2-Fc positive RBCs.

As shown in FIG. 6, ACE2-Fc labeled RBCs not only showed the same lifespan as that of the control groups (mice transfused with RBCs without ACE2-Fc-LPETG tag), but also exhibited sustained signals in circulation for 28 days.

### ACE2-RBC Inhibition of SARS-CoV-2 Virus

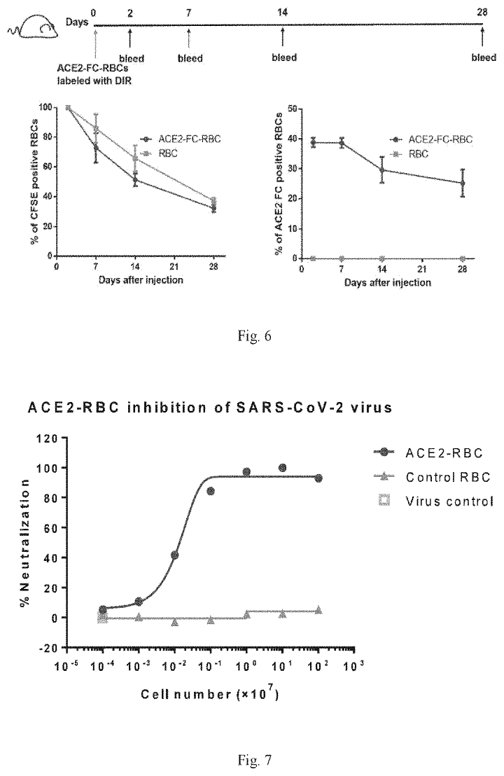

The control RBC or ACE2-Fc-RBC was serially diluted and incubated with SARS-COV-2 virus for 1 hour. The supernatant was centrifuged and used to infect VERO-E6 cells for 48 hours. Fluorescence quantitative PCR was used to detect the level of virus infection and analyze virus neutralization ability. The results in FIG. 7 showed a dose-dependent virus neutralization ability of ACE2-Fc-RBC.

The results in FIGS. 5-7 showed that this method can effectively label ACE2-Fc to the surface of RBC, and showed a dose-dependent virus neutralization ability.

### REFERENCES

[1] J. W. Yoo, D. J. Irvine, D. E. Discher, and S. Mitragotri, “Bio-inspired, bioengineered and biomimetic drug delivery carriers,” Nat. Rev. Drug Discov., vol. 10, no. 7, pp. 521-535, 2011.
[2] J. M. Antos, J. Ingram, T. Fang, N. Pishesha, M. C. Truttmann, and H. L. Ploegh, “Site-Specific Protein Labeling via Sortase-Mediated transpeptidation,” 2017.
[3] J. Shi, L. Kundrat, N. Pishesha, A. Bilate, C. Theile, and T. Maruyama, “Engineered red blood cells as carriers for systemic delivery of a wide array of functional probes,” pp. 1-6, 2014.
[4] P. Daniel Harris, BA, Lynn McNicoll, MD, Gary Epstein-Lubow, MD, and Kali S. Thomas, “Recent Advances in Sortase-Catalyzed Ligation Methodology,” Physiol. Behav., vol. 176, no. 1, pp. 139-148, 2017.
[5] Y. Ge, L. Chen, S. Liu, J. Zhao, H. Zhang, and P. R. Chen, “Enzyme-Mediated Intercellular Proximity Labeling for Detecting Cell-Cell Interactions,” J. Am. Chem. Soc., vol. 141, no. 5, pp. 1833-1837, 2019.
[6] Y. Zhu, T. Guo, and S. K. Sze, “Chapter 22 Elucidating Structural Dynamics of Integral Membrane Proteins on Native Cell Surface by Hydroxyl Radical Footprinting and Nano LC-MS/MS,” vol. 790, pp. 287-303.
[7] Swee, L. K.; Lourido, S.; Bell, G. W.; Ingram, J. R.; Ploegh, H. L. One-step Enzymatic Modification of the Cell Surface Redirects Cellular Cytotoxicity and Parasite Tropism. ACS Chem. Biol. 2015, 10, 460-465.
[8] N. Pishesha et al., “Engineered erythrocytes covalently linked to antigenic peptides can protect against autoimmune disease,” vol. 114, no. 17, 2017.
[9] A. J. Grimm, S. Kontos, G. Diaceri, and X. Quaglia-thermes, “Memory of tolerance and induction of regulatory T cells by erythrocyte-targeted antigens,” Nat. Publ. Gr., pp. 1-11.
[10] A. Karaplis, L. Pong, L. Chien, and N. Chattopadhyay, “Parathyroid hormone ablation alters erythrocyte parameters that are rescued by calcium-sensing receptor gene deletion,” vol. 91, no. 1, pp. 37-45, 2014.
[10] Kuba K, Imai Y, Rao S, Gao H, Guo F, et al. 2005. Nat Med 11: 875-9.
[11] Glowacka I, Bertram S, Herzog P, et al. 2010. Journal of Virology 84: 1198-205.
[12] Huang F, Guo J, Zou Z, Liu J, Cao B, et al. 2014. Nat Commun 5: 3595.
[13] Imai Y, Kuba K, Rao S, Huan Y, Guo F, et al. 2005. Nature 436: 112-6.

1. A red blood cell (RBC) having an agent linked thereto, wherein the agent is linked to at least one endogenous, non-engineered membrane protein of the RBC by a sortase-mediated reaction, wherein the sortase-mediated reaction comprises a sortase-mediated glycine conjugation and/or a sortase-mediated lysine side chain ε-amino group conjugation.

2. The red blood cell of claim 1, wherein the sortase-mediated glycine conjugation and/or the sortase-mediated lysine side chain ε-amino group conjugation occur at least on glycine₍ₙ₎ and/or lysine ε-amino group at internal sites of the extracellular domain of the at least one endogenous, non-engineered membrane protein, optionally n being 1 or 2.

3. The red blood cell of claim 1, wherein the RBC has not been genetically engineered to express a protein comprising a sortase recognition motif or a nucleophilic acceptor sequence, and optionally the RBC is a natural RBC.

4. (canceled)

5. The red blood cell of claim 1, wherein the sortase is a Staphylococcus aureus transpeptidase A variant (mgSrtA), optionally the mgSrtA comprising an amino acid sequence having at least 60% identity to the amino acid sequence as set forth in SEQ ID NO: 3.

6. The red blood cell of claim 5, wherein the mgSrtA comprises the amino acid sequence as set forth in SEQ ID NO: 3 or SEQ ID NO: 27.

7. The red blood cell of claim 1, wherein the agent, before being linked to the RBC, comprises a sortase recognition motif on its C-terminus, and optionally wherein the sortase recognition motif comprises an amino acid sequence selected from the group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid.

8. (canceled)

9. The red blood cell of claim 1, wherein the agent comprises a binding agent, a therapeutic agent, or a detection agent, optionally the agent comprises a peptide comprising an extracellular domain of oligomeric ACE2.

10. The red blood cell of claim 1, wherein the at least one endogenous, non-engineered membrane protein on the surface of the RBC comprises a structure of A1-LPXT-P1, in which LPXT is linked to a glycine₍ₙ₎ in P1, and/or a structure of A1-LPXT-P2, in which LPXT is linked to the side chain ε-amino group of lysine in P2, wherein n is 1 or 2, A1 represents the agent, P1 and P2 independently represent the extracellular domain of the at least one endogenous, non-engineered membrane protein, and X represents any amino acids.

11.-12. (canceled)

13. A method for covalently modifying at least one endogenous, non-engineered membrane protein of a red blood cell (RBC), comprising contacting the RBC with a sortase substrate that comprises a sortase recognition motif and an agent, in the presence of a sortase under conditions suitable for the sortase to conjugate the sortase substrate to the at least one endogenous, non-engineered membrane protein of the RBC by a sortase-mediated reaction, wherein the sortase-mediated reaction comprises a sortase-mediated glycine conjugation and/or a sortase-mediated lysine side chain ε-amino group conjugation.

14. The method of claim 13, wherein the sortase-mediated glycine conjugation and/or the sortase-mediated lysine side chain ε-amino group conjugation occur at least on glycine₍ₙ₎ and/or lysine ε-amino group at internal sites of the extracellular domain of the at least one endogenous, non-engineered membrane protein, optionally n being 1 or 2.

15. The method of claim 13, wherein the RBC has not been genetically engineered to express a protein comprising a sortase recognition motif or a nucleophilic acceptor sequence, and optionally the RBC is a natural RBC a natural human RBC.

16. (canceled)

17. The method of claim 13, wherein the sortase is a Staphylococcus aureus transpeptidase A variant (mgSrtA), optionally the mgSrtA comprising an amino acid sequence having at least 60% identity to the amino acid sequence as set forth in SEQ ID NO: 3.

18. The method of claim 17, wherein the mgSrtA comprises the amino acid sequence as set forth in SEQ ID NO: 3 or SEQ ID NO: 27.

19. The method of claim 13, wherein the sortase substrate comprises the sortase recognition motif on its C-terminus, and optionally the sortase recognition motif comprises an amino acid sequence selected from the group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS and LPXTA, wherein X is any amino acid.

20. (canceled)

21. The method of claim 13, wherein the agent comprises a binding agent, a therapeutic agent, or a detection agent, optionally the agent comprises a peptide comprising an extracellular domain of oligomeric ACE2.

22. The method of claim 13, wherein the covalently modified at least one endogenous, non-engineered membrane protein on the surface of the RBC comprises a structure of A1-LPXT-P1, in which LPXT is linked to a glycine₍ₙ₎ in P1, and/or a structure of A1-LPXT-P2, in which LPXT is linked to the side chain ε-amino group of lysine in P2, wherein n is 1 or 2, A1 represents the agent, P1 and P2 independently represent the at least one endogenous, non-engineered membrane protein, and X represents any amino acids.

23. (canceled)

24. A composition comprising the red blood cell of claim 1 and optionally a physiologically acceptable carrier.

25. A composition comprising a sortase, a sortase substrate that comprises a sortase recognition motif and an agent, and optionally a physiologically acceptable carrier, wherein, optionally upon contacting red blood cells (RBC) in vivo, the sortase is capable of mediating a glycine₍ₙ₎ conjugation and/or a lysine side chain ε-amino group conjugation at internal sites of the extracellular domain of at least one endogenous, non-engineered membrane protein of a RBC, optionally n being 1 or 2,
optionally wherein the sortase is a Staphylococcus aureus transpeptidase A variant (mgSrtA), optionally wherein the mgSrtA comprises the amino acid sequence as set forth in SEQ ID NO: 3 or in SEQ ID NO: 27,
further optionally wherein the at least one endogenous, non-engineered membrane protein conjugated with the sortase substrate comprises a structure of A¹-LPXT-P¹, in which LPXT is linked to a glycine₍ₙ₎ in P¹, and/or a structure of A¹-LPXT-P², in which LPXT is linked to the side chain ε-amino group of lysine in P², n is 1 or 2, A¹ represents the agent, P¹ and P² independently represent the at least one endogenous, non-engineered membrane protein, and X represents any amino acids.

26.-33. (canceled)

34. The composition of claim 25, wherein the sortase has been further modified to enhance its stabilization in circulation and/or reduce its immunogenicity, optionally wherein the sortase has been PEGylated and/or linked to an Fc fragment.

35. (canceled)

36. A method for diagnosing, treating or preventing a disorder, condition or disease in a subject in need thereof or for delivering the agent to a subject in need thereof, comprising administering the red blood cell of claim 1 to the subject.

37. The method of claim 36, wherein the disorder, condition or disease is selected from a group consisting of tumors or cancers, metabolic diseases, bacterial infections, virus infections, autoimmune diseases and inflammatory diseases, optionally wherein the virus infection is SARS-COV or SARS-COV-2 infection.

38. (canceled)

39. The method of claim 13, wherein the method increases the circulation time or plasma half-life of the agent in a subject.

40.-46. (canceled)
